(19) United States
(12) Patent Application Publication
(10) Pub. No.: US 2026/0125745 A1
(43) Pub. Date: May 7, 2026

(54) BIOLOGICAL BASED WIRING OF ELECTRONIC CIRCUITS FOR MOLECULAR SENSING APPLICATIONS

(71) Applicant: Atlas Data Storage, Inc., South San Francisco, CA (US)

(72) Inventor: Michael Shane BOWEN, Encinitas, CA (US)

(21) Appl. No.: 19/383,197

(22) Filed: Nov. 7, 2025

Related U.S. Application Data
(60) Provisional application No. 63/717,727, filed on Nov. 7, 2024.
(60) Provisional application No. 63/764,479, filed on Feb. 27, 2025.

(51) Int. Cl. C12Q 1/6825

(52) U.S. Cl. CPC C12Q 1/6825

(57) ABSTRACT

In some embodiments, an apparatus comprises a first electrode and a second electrode defining a gap therebetween; a molecular wire disposed across the gap, and configured to electrically connect the first electrode and the second electrode, the molecular wire including: at least one molecule of deoxyribonucleic acid (DNA) including a double stranded portion; and a plurality of functional groups coupled to nucleotides of the at least one molecule of DNA at predetermined positions along the at least one molecule of DNA, the plurality of functional groups configured to cause electron delocalization along the molecular wire to increase an electrical conductivity of the molecular wire; and a first molecular linker configured to couple a first portion of the molecular wire to the first electrode and a second molecular linker configured to couple a second portion of the molecular wire to the second electrode.

### CROSS-REFERENCE TO RELATED APPLICATIONS

This application claims priority to and the benefit of U.S. Provisional Application No. 63/717,727, filed Nov. 7, 2024, and entitled “Biological Based Wiring of Electronic Circuits for Molecular Sensing Applications,” and U.S. Provisional Application No. 63/764,479, filed Feb. 27, 2025, and entitled “Biological Based Programmable Arrays,” the disclosure of each of which is incorporated by reference herein in its entirety.

### TECHNICAL FIELD

Embodiments described herein relate to molecular sensors including biological wires. More specifically, the embodiments described herein relate to DNA based wires for molecular sensing applications.

### BACKGROUND

Molecular sensors can be used for many applications including affinity binder-based proteomics (e.g., protein identification and quantification) via aptamers or antibodies, Enzyme linked immunosorbent assay (ELISA) type measurements of proteins (e.g., using affinity binders to form sandwich assays), Genotyping, deoxyribonucleic acid (DNA) sequencing, DNA decoding, and/or DNA data storage. Current approaches include use fluorescence to indicate the occurrence of a molecular event (e.g., binding of an affinity binder to a probe or incorporation of a base into DNA) or use of nanopores for electronic readout. However, these approaches have limitations around throughput, cost, and ability to reprogram the platform for different uses.

### SUMMARY

In some embodiments, an apparatus comprises a first electrode and a second electrode defining a gap therebetween; a molecular wire disposed across the gap, and configured to electrically connect the first electrode and the second electrode, the molecular wire including: at least one molecule of deoxyribonucleic acid (DNA) including a double stranded portion; and a plurality of functional groups coupled to nucleotides of the at least one molecule of DNA at predetermined positions along the at least one molecule of DNA, the plurality of functional groups configured to cause electron delocalization along the molecular wire to increase an electrical conductivity of the molecular wire; and a first molecular linker configured to couple a first portion of the molecular wire to the first electrode and a second molecular linker configured to couple a second portion of the molecular wire to the second electrode.

In some embodiments, an apparatus comprises: an electrode; a molecular wire configured to be coupled to the electrode to conduct an electrical signal to and from the electrode, the molecular wire including at least one molecule of deoxyribonucleic acid (DNA) including a double stranded portion; and a linker disposed on the electrode and configured to couple the molecular wire to the electrode, the linker including: a DNA origami structure including a plurality of binding sites, the plurality of binding sites including at least one binding site including a first DNA sequence configured to hybridize to a binding region of the molecular wire having a second DNA sequence complementary to the first DNA sequence.

In some embodiments, an apparatus comprises a semiconductor chip including a plurality of electrodes; a plurality of molecular wires, each molecular wire of the plurality of molecular wires configured to electrically connect a set of electrodes of the plurality of electrodes to form one or more circuits, the plurality of molecular wires including at least one deoxyribonucleic (DNA) molecule; and a plurality of molecular linkers disposed on the plurality of electrodes, the plurality of molecular linkers configured to attach a respective portion of each molecular wire of the plurality of molecular wires to a respective electrode of the plurality of electrodes, at least a portion of the plurality of molecular wires configured to be replaced via toehold strand displacement such that the one or more circuits formed by the plurality of molecular wires can be regenerated or reprogrammed.

In some embodiments, a system comprises a semiconductor chip including a plurality of molecular circuits, each molecular circuit of the plurality of molecular circuits including: a pair of electrodes defining a gap therebetween; a molecular wire disposed across the gap and configured to electrically connect the pair of electrodes, the molecular wire including at least one molecule of deoxyribonucleic (DNA); and a polymerase including a binding site separate from an active portion of the polymerase, the binding site configured to bind to a portion of the molecular wire, the polymerase configured to undergo a conformation change in response to interacting with a nucleotide of a target strand of DNA to be sequenced, the conformation change configured to cause a change in a level of current directed between the pair of electrodes; and a processor operatively coupled to the semiconductor chip, the processor configured to: receive, as polymerase sequentially interacts with nucleotides of the target strand of DNA to be sequenced, signals corresponding to the level of current directed between the pair of electrodes; and determine a sequence of the target strand of DNA based on the signals.

### BRIEF DESCRIPTION OF THE DRAWINGS

FIG. 1 shows a schematic diagram of a molecular circuit including a molecular wire, according to some embodiments.

FIG. 2A shows an illustration of a DNA-based wire configured to couple a first electrode and a second electrode, according to an embodiment.

FIG. 2B shows an enlarged view of a first foundation oligo of the DNA-based wire of FIG. 2A.

FIG. 3 shows a bridge body oligo of a DNA-based wire including a first end configured to hybridize to a first foundation oligo and a second end configured to hybridize to a second foundation oligo, according to an embodiment.

FIG. 4 shows the DNA-based wire including a bridge body compliment hybridized to the bridge body oligo to create a double stranded DNA, thereby completing a circuit between a pair of electrodes, according to an embodiment.

FIG. 5 shows a bridge body complement formed from a plurality of short oligo strands each short oligo strand including a toehold region, according to an embodiment.

FIG. 6 shows a DNA-based wire including a genotyping sensor region, according to an embodiment.

FIG. 7 shows a DNA-based wire including a pair of bridge junction arms configured to be coupled to a functional probe, according to an embodiment.

FIG. 8 shows the DNA-based wire of FIG. 7 including the functional probe coupled to an end portion of the bridge junction arms, according to embodiments.

FIG. 9A shows a DNA-based wire including a functional probe coupled to bridge junction arms that are not complementary to one another, according to an embodiment.

FIG. 9B shows the DNA-based wire of FIG. 9A including an apex bridge configured to control an amount of current through the functional probe, according to an embodiment.

FIG. 10 shows an example of toehold mediated strand displacement, according to an embodiment.

FIGS. 11A-11D show different open circuit configurations of a first electrode terminal and a second electrode terminal, according to embodiments.

FIG. 12A shows electrode terminals with linkers extending therefrom, and FIG. 12B shows a bridge oligo connecting the linkers, according to embodiments.

FIG. 13 shows a molecular circuit including three electrode terminals and a catalytic operator, according to some embodiments.

FIG. 14 shows a molecular circuit including a plurality of electrode terminals and a plurality of catalytic operators.

FIG. 15 is a schematic block diagram of an example method of building a molecular circuit, according to an embodiment.

### DETAILED DESCRIPTION

Embodiments described herein relate to systems (e.g., molecular sensing systems), devices, and methods configured to detect molecular events. Molecular sensors can be useful for many applications including affinity binder-based proteomics (e.g., protein identification and quantification) via aptamers or antibodies, ELISA type measurements of proteins (e.g., using affinity binders to form sandwich assays), protein sequencing, genotyping, DNA sequencing, DNA decoding, and/or DNA data storage. Molecular sensing enables measurement of changes in electrical properties of a circuit that are induced by one or more stimulating events. Stimulating events can include, for example, a change in a single molecule or an interaction of several molecules. Current approaches that leverage electronic properties to sense chemical events include: using pH sensing device (ISFETs), monitoring the current through a biological or synthetic nanopore, monitoring perturbations to a current flowing through an engineered wire, monitoring changes in response curves of a transistor that includes molecular probes immobilized on the gate region of the device, and/or converting optical stimulation into electronic signals using an optical sensor (e.g., CMOS).

Existing approaches to molecular sensing (e.g., using nanopores or protein-based wires) suffer from limitations around size of the devices, lack of ability to precisely control conductivity of the wire, inability for sensor regeneration, and/or have limitations in terms of precision and scalability.

Embodiments described herein address the drawbacks of current sensing, sequencing, and synthesis systems by integrating one or more molecular wires (i.e., nanostructured components) synthesized out of nucleic acid-based polymers (i.e. DNA, RNA, PNA, etc.). In some embodiments, the molecular wires may optionally be integrated with (e.g., conjugated to) one or more functional groups and/or proteins (e.g., transduction agents). Embodiments described herein include a method for constructing a circuit, or a portion of a circuit out of predetermined collections of individual molecules. DNA is a polymer that can be deterministically engineered to have specific sequences and/or can be modified to influence its physical (e.g., electrical and/or thermal) and chemical properties. In some embodiments, the molecular wire may include a plurality of strands of DNA (e.g., a plurality of oligonucleotides (“oligo(s)”)), and therefore the molecular wire may be referred to herein as a nanostructured device.

In some embodiments, the integrated molecular wires may be coupled to conventional materials and/or components used to construct integrated circuits. For example, the integrated molecular wires may be coupled to nonbiological conductors, insulators and semiconductors such as copper, ruthenium, titanium, titanium oxide, titanium oxy-nitride, gold, platinum, palladium, silicon, silicon dioxide, hafnium oxide, graphene, carbon nanotubes, etc. These conventional materials may include materials that can be routinely used in the fabrication of integrated circuits and/or optical sensors. In some embodiments, a conventional circuit may be opened to create a physical gap (e.g., an opening, space, aperture) between a pair of electrodes, which halts or substantially slows or inhibits the functioning of at least a portion of the circuit. The circuit in the open state may have a current flow therethrough that is a function of one or more factors such as, for example, a composition of a buffer solution surrounding the open circuit. The circuit may then be closed (e.g., an electrical connection may be formed across the gap) with a collection of molecular wires, engineered from one or more biological-based materials such as DNA. The circuit in the closed state may have a current flow therethrough that is a function of the molecular wire, for example, electrical properties of the molecular wire such as resistivity, conductivity, etc. The current flow in the closed state may be larger than the current flow in the open state. In some embodiments, a functional probe (e.g., a polymerase) may be integrated into the circuit such that the circuit when the functional probe is in a first configuration or conformation has a first current flow and the circuit when the functional probe is in a second configuration or conformation has a second current flow greater than the first current flow. In some embodiments, the first current flow when the functional probe is in the first configuration can be similar to the current flow when the circuit is in the open state.

The flow of electrons through a circuit is governed by several factors, including the physical properties of the materials used to fabricate the circuit, the surrounding media, and any stimuli that are either ambiently or intentionally subject to interacting with components of the circuit. Sensors (e.g., electronic sensors) can be formed by designing circuits with precisely tuned properties, such as conductivity, impedance, resistivity to current, or capacitance to voltage, and monitoring the specific or collective properties of the device upon exposure to stimuli/events being monitored. A signal can be detected, for example, by observing changes in the current flow or resistivity through the circuit or an electric potential shift in a capacitor.

An electronic pathway is the trajectory through which electrons (or holes) flow through a material. The flow of electrons (or holes) through a circuit is referred to as a current and is induced when an electrical potential is applied to the circuit (i.e. a battery is hooked up to the terminals of the circuit). Embodiments described herein can include a nucleic acid polymer that forms a module within the circuit, specifically a molecular wire, and defines a constrained trajectory of the current. The molecular wire provides and engineered pathway for tuning the performance of the circuit, resulting in a precisely engineered sensor. Modules in a circuit can include resistors, capacitors, amplifiers and the like. In some instances, the molecular wire can be analogous to a variable resistor where the variability, as a function of time, is the signal that can be monitored to form a basis of a sensing action.

In some embodiments, the systems and devices herein may sense molecular events by measuring changes in electronic properties of the circuit. For example, the systems and devices herein may monitor a current passing therethrough and/or a voltage change across a set of electrode terminals induced through the presence, or lack thereof, of molecule(s) being probed by the molecular wire

The molecular sensors described herein may provide many advantages including, for example, improved sensitivity (e.g., single molecule sensitivity), improved signal to noise ratio (SNR), increased rate of data acquisition, increased density of sensors in a given surface area, an increased dynamic range (facilitated by enabling a very large number of measurements in a short period of time), improved reproducibility, and a low cost of manufacturing relative to existing sensing technologies. The sensors described herein contribute to advancements in (1) integrated circuit design, (2) microfluidic device fabrication, (3) advanced materials (4) molecular electronics, (5) nanoengineering / molecular nanostructures, and (6) surface regeneration.

Embodiments described herein may include methods for in-situ generation of the molecular arrays. Embodiments described herein may include methods for regeneration of the molecular wire/nanostructured component of the sensor following use. Regeneration of the molecular wire can enable usage of the sensor for more than one application, thereby decreasing cost, increasing device lifetime, and increasing efficiency in workflow.

Molecular sensing can be particularly useful for DNA sequencing and/or DNA synthesis. Current DNA sequencing technologies have advanced significantly, but also face challenges in speed, cost, and accuracy. DNA synthesis is typically performed using phosphoramidite and classical enzymatic methods. Both methods are slow and generally require continuous solution refreshing. Additionally, there are different multiplexing techniques such as electrochemical, light-based, and inkjet-based multiplexing.

Embodiments described herein use self-assembled molecular electronics configured to perform DNA synthesis and/or sequencing using protein design tools, the activity of which can be controlled and read out directly using electrical signals. Embodiments described herein integrate biological-based materials into conventional integrated circuits can facilitate large-scale fabrication of sensor arrays (e.g., biological based programmable arrays). In some embodiments, a sensor array may include a plurality of sensors configured to function in parallel, thereby enabling parallel detection of a large number (e.g., 1s (<10 ), to 100s, to 1000s, to millions to hundreds of millions) of molecular events in parallel (e.g., simultaneously). These molecular events can be on single molecules, such as DNA or an individual protein, or on colonies of molecules such as clusters (e.g., polonys) of identical molecules, such as a collection of similar strands of DNA or proteins. In some embodiments, a single sensor can detect a plurality of molecular events. In some embodiments, sensors within a specific, packaged device can be customized to a specific application by adjusting a sequence within the DNA wire, a type and/or frequency of functional groups or moieties integrated into the DNA wire, and/or a structure or configuration of the oligonucleotide strands in the DNA wire. In some embodiments, a device can include a single sensor. In some embodiments, the device can include a plurality of sensors.

Embodiments described herein can include a biological-based programmable sensing array. In some embodiments, the biological-based programmable sensing array can be analogous to a Field Programmable Gate Array (FPGA) that enables a user to reconfigure hardware to meet specific use case requirements. For example, the systems and devices herein can include molecular architectures that can be user defined and created in real-time based on electrically directed nanoscale control of single molecular catalysts. With a conventional FPGA, customization is accomplished through a user defined software update, written in a Hardware Description Language (HDL) and uploaded (“flashed”) to the hardware. The biological-based programmable arrays described herein can enable customization through introduction of a user defined set of reagents. This reagent set can be formulated upon a base molecular foundation, leveraging biomolecules such as nucleic acid polymers and enzymes, that can enable integration into the hardware circuitry.

In some embodiments, applications for biological based programable arrays include: i) precise control of multi-step catalytic reactions (i.e., molecular synthesis), ii) molecular sensing in genomics, proteomics and metabolomics (i.e., sequencing of nucleic acids and/or proteins, and/or identification and quantification of analytes of interest), iii) therapeutic development (e.g., synthesis of mRNA), and iv) logic operations applicable to molecular electronics for future iterations of computer hardware. Industrial applications that are not sufficiently addressed with currently available technology and that can benefit immediately from these advancements include i) data storage (i.e., rapid and low-cost synthesis and sequencing of nucleic acids to support DNA-based data storage solutions), and ii) rapid development of advanced therapeutics in personalized medicine. Both of these applications use rapid cycles of learning during the technology development phase and it is desirable to conduct these at a sufficiently low price point. These two applications have common desired parameters to rapidly synthesize and sequence nucleic acid polymers and can serve as the model systems. Realization of a biological-based programmable array can dramatically accelerate advancement in the aforementioned, as well as many other relevant fields.

Embodiments described herein may include chip-based technologies that enable micro and/or nano-electronic control of enzymes to both synthesize and sequence DNA at the single molecule level at high throughput, within the same device or system and using a water-based wash-free chemistry. In some embodiments, the systems and devices described herein can be configured to perform both DNA sequencing and DNA synthesis using one or more molecular circuits on a semiconductor chip. This represents a significant breakthrough in biotechnology, particularly for DNA storage applications. Firstly, the integration of synthesis and reading capabilities on a single platform is a game-changer for DNA data storage. DNA, with its high density and long-term stability, is an ideal medium for storing vast amounts of data. With current solutions for synthesis and sequencing technologies, the application of data archiving (cold storage) is achievable.

To take advantage of all the upsides available from DNA as the storage medium, it is desirable to integrate write (e.g., synthesis) and read (e.g., sequencing) processes and push the technology into use cases where the time between writing and reading becomes more coincident. The systems, devices, and methods described herein facilitate the ability to precisely synthesize and accurately read DNA sequences, in a single platform, making it highly suitable for encoding digital information into DNA and leveraging this across several use cases. The one-pot solution simplifies the process, making it more efficient, readily scalable, easily decentralizable and cost-disruptive. Conventional methodologies for both synthesis and sequencing by synthesis (SBS) of nucleic acid polymers and/or phosphonamidite, for example, are generally based on cyclic processes that leverage highly caustic and environmentally hazardous chemicals. These processes are cyclic in nature and generally employ extensive cleavage and wash steps between each incorporation or read steps, which results in large volumes of reagent consumption with cycle durations that decrease the polymerase incorporation rate by 3 orders of magnitude. In contrast, embodiments described herein include chemical processes that are enzymatic based, performed in aqueous conditions, and leverage electronically actuated reactions and/or changes in electrical properties caused by enzymatic activity, which allow for the elimination of the cyclic wash steps and enable a single-pot reaction chemistry to be used. This can revolutionize data storage, providing a sustainable and scalable alternative to traditional storage methods such as magnet tape and hard disk drive technologies.

Secondly, the use of nano-electronic control allows for precise manipulation of polymerase activity, which is crucial for maintaining the integrity of stored data. High-resolution control over DNA synthesis ensures that the encoded information is accurate and reliable. Systems, devices, and methods described herein can perform single molecule synthesis, amplification, and reading. The density of the sensing elements described herein (e.g., 250 nm pitch) is not achievable with optical based solutions.

Systems, devices, and methods described herein can also enable super Poisson loading of microfabricated electrodes through DNA origami placement (i.e., a very high probability of desired placement based on a Poisson distribution). Currently there is no solution to placing single molecules with a nanometer scale resolution on surfaces, for example, conductive surfaces. Current techniques can include coating a bead (particle) with a colony of molecules and loading those particles into wells (e.g., an array of well plates or silicon plates), and loading small wells (zero mode waveguides) with complexes of molecules (e.g., ink-jet printing reagents in the case of phosphonamidite-based synthesis of nucleic acids). However, this technique still loads with a Poisson distribution and super Poisson loading is generally not achievable.

Embodiments described herein can achieve single molecule placement within nanometer resolution by using DNA origami that can act as a scaffold upon which the molecular circuits are constructed. For example, DNA origami (when used as a linker to the electrode and/or as the molecular wire) can enable localization of particular molecular circuitry within a semiconductor chip. Therefore, the foundational architecture of the devices described herein is based on deterministic placement of specific molecules, enabled through the use of DNA nanotechnology (e.g., DNA origami). Determinism can be facilitated through the use of base-pairing intrinsic to DNA / RNA. Programmability can be facilitated through the conjugation of nucleic acids to larger molecules such as proteins. Embodiments described herein can have tunable electrical performance, having the ability to be engineered, through the use of functional groups or ligands integrated with individual nucleotides (e.g., for building resistors, capacitors, switches, rectifiers, etc.)

The embodiments described herein can lead to the development of advanced DNA storage systems that can store exabytes of data in a compact and durable format, addressing the growing demand for data storage in the digital age. In current systems, reading a single terabyte (TB) of data generally includes reading of 50 terabases, which is the equivalent of 500 human genomes, resulting in inaccessibly high price (e.g., about $50 k per terabyte). The systems, devices, and methods described herein can enable reading and storage of large amounts of data. In some embodiments, the systems, devices, and methods described herein can reduce cost by an order of magnitude (e.g., to approximately $5,000 per terabyte), two orders of magnitude (e.g., to approximately $500 per terabyte), or three orders of magnitude (e.g., to approximately $50 per terabyte). Additionally, the systems devices and methods here can decrease an amount of time to sequence for DNA storage applications. Current systems take between 50 hours to 150 hours to sequence 1 terabyte of data using DNA sequencing. The embodiments described herein can sequence a terabyte of data in less than about 120 minutes, in less than about 60 minutes, in less than about 30 minutes. Embodiments described herein can sequence a terabyte of data in less than about 30 minutes. Embodiments, described herein can achieve throughput of greater than 1,000 bases per second per sensor and have sensor spacing ranging from about 5 μm down to 250 nanometers (nm).

Beyond enabling DNA data storage, embodiments of the systems and methods described herein can have significant environmental implications. The data storage industry is one of the fastest growing industries on the planet, which is attributed to the data growth associated with health, insurance, security, government, entertainment, critical communications, and advancements in artificial intelligence (AI). However, the existing, insufficient capacity and expense associated with today's storage solutions are constraining the ability to retain the data being generated. The relatively poor density and durability of existing solutions result in the generation of tremendous amounts of CO₂, carbon consumption, and e-waste, all of which continue to grow as we generate more data every day. With a full transition to DNA-based data storage, it is estimated that there will be more than 100× reduction in CO₂ emission than tape and more than 1,200× less than hard disk drives.

In addition to DNA storage, embodiments described herein can have significant implications for customized, delocalized pathogen detection and vaccine production. The ability to rapidly identify specific antigen targets, synthesize and read DNA on the same platform enables the swift development and production of response plans such as vaccines tailored to specific pathogens. This is particularly valuable in responding to emerging infectious diseases, where time is of the essence. The compact and integrated nature of the embodiments described herein can allow for its deployment in various settings, including remote and resource-limited areas, facilitating localized vaccine production and distribution.

Moreover, embodiments described herein can include a controlled enzymatic chip to enable synthesis on demand of other drugs and compounds for sensing (bio)chemicals, without depending on complex supply chains.

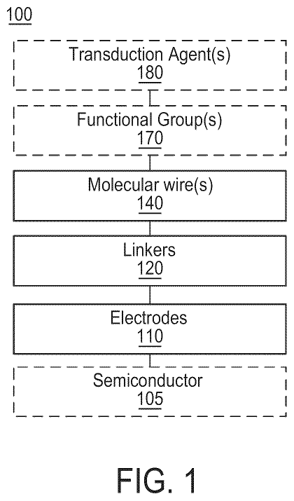

FIG. 1 shows a schematic block diagram of a molecular circuit device 100 (hereinafter, “device 100”), according to some embodiments. As shown, the device 100 can include one or more molecular wires 140 configured to be coupled to one or more electrodes 110 via one or more linkers 120 to form one or more molecular circuits. In some embodiments, the device 100 can include a pair of electrodes including a first electrode and a second electrode defining a gap (e.g., a nanogap) therebetween. The molecular wire 140 can be disposed across the gap and configured to electrically connect the first electrode and the second electrode. In some embodiments, the one or more linkers 120 can include a first linker configured to attach a first portion of the molecular wire 140 to the first electrode and a second linker configured to attach a second portion of the molecular wire 140 to the second electrode. In some embodiments, the electrodes 110 can be disposed on a semiconductor chip 105 (e.g., fabricated for a particular use) such that when the molecular wire 140 is attached to the electrodes 110, the molecular wire 140 is incorporated into pre-programmed circuitry of the semiconductor chip 105.

In some embodiments, the molecular wire 140 can be formed from or include nucleic acid-based polymers (i.e. DNA, RNA, PNA, etc.). In some embodiments, the molecular 140 can be formed from or include one or more polypeptides and/or proteins. In some embodiments, the molecular wire 140 can be formed from or include at least one molecule of DNA. In some embodiments, the molecule of DNA can include at least one double stranded portion. In some embodiments, when the molecular wire 140 is coupled to the first electrode and the second electrode via the one or more linkers 120, an entire length of the molecule of DNA may be double stranded. Double stranded DNA (“dsDNA”) is capable of conducting electricity and is therefore a suitable option for forming molecular wires for molecular sensing applications. Alternatively, single stranded DNA (“ssDNA”) has poor conductivity. One way through which conductivity of a DNA strand can be reduced (e.g., resistivity can be increased) is through single base mismatching. Therefore, for a dsDNA (i.e., a duplex) to serve as a conductor, a number of base mis-matches in the molecular wire, or a number of base mismatches in the duplex should be limited (e.g., be about zero). Conversely, if conductivity of a duplex is to be decreased for a particular application, the duplex can be sequenced to include a predetermined number of base mismatches.

DNA is formed of sequences of nucleotides including guanine (“G”), adenine (“A”), thymine (“T”) and cytosine (“C”). The sequence of DNA strand(s) and/or functional groups 170 integrated into (or conjugated to) the DNA strand(s) that form a nanostructure can contribute to one or more properties of the nanostructure such as a shape or structure of the nanostructure, thermal stability, conductivity, impedance, resistivity to current, and/or capacitance to voltage of the nanostructure. In some embodiments, the molecular wire 140 may include duplexes that are B-DNA, meaning that the oligos include collections of all four nucleotides and upon the oligos forming a duplex, form a right-handed double helix. A helix width (or diameter) is a maximum lateral width of the helix structure. The helix width of B-DNA is about 2 nanometers (nm), and there are 10 base pairs in each turn of the helix, with each turn of the helix having a length of about 3.4 nm. The distance between adjacent deoxyribonucleotides is about 0.34 nm. B-DNA may be preferable to Z-DNA, which includes oligos having long (CG)n and (TG)n stretches, which may be undesirable for reasons described in further detail below. In some embodiments, the molecular wire 140 may include nucleic acid junctions to assemble a stable nanostructure.

In some embodiments, the electrodes 110 may include any suitable material such as, for example, gold, platinum, ruthenium, titanium, titanium-oxide, titanium oxy-nitride, indium tin oxide (ITO), iridium, alloys thereof, or a suitable combination of these materials. In some embodiments, the electrodes can be constructed from layers of different materials such as, for example, platinum and titanium or platinum and titanium oxy-nitride. In some embodiments, the electrodes 110 may be fabricated by capping a conductive metal with an oxide. In some embodiments, the electrodes 110 may be fabricated out of a conductive metal oxide such as indium tin oxide (ITO) or titanium oxy-nitride. One advantage of including a conductive metal oxide in the electrodes 110 is that the oxide facilitates covalent linkage of surface chemistries, such as silanes, whereas gold-thiol linkages are not covalent. In some embodiments, the electrodes 110 can include gold deposited on a titanium adhesion layer on silicon dioxide (i.e., the “conventional” materials used to fabricate circuits). In some embodiments, the 3′end of each of the single stranded oligos that made up the duplex of the molecular wire 140 can be functionalized with a linker 120 (e.g., with a thiol group or a silane for the case when a conductive oxide is used) to facilitate covalent or quasi-covalent bonding of the molecular wire 140 to each electrode 110 (e.g., gold electrode), described in further detail below. By having the 3′ ends functionalized on both of the complementary oligos which make up the duplex of the molecular wire 140, there is thiol functionality on both ends of duplex that enables attachment of each end of the duplex to a respective one of the electrodes 110 such that the duplex forms the molecular bridge between the two electrodes 110. Additionally or alternatively, a functional group on one or both ends of the oligos of the duplex can be clicked to an alkyne terminated silane that has been deposited onto a metal oxide electrode.

There are different approaches to controlling conductivity of dsDNA including: i) integration of ligands (e.g., phenol groups covalently bound to nucleotides), ii) integration of redox reporters (e.g., methylene blue covalently bound to nucleotides), iii) integration of intercalating redox groups into the double stranded backbone, and/or iv) constructing the molecular wire 140 from DNA based nanostructures such as DNA origami.

In some embodiments, the molecular wire 140 can include a plurality of DNA molecules (e.g., modified DNA molecules) to increase conductivity between the electrodes 110. For example, the molecular nanowire 140 can be constructed using DNA origami techniques, which allows for the precise placement of molecules at the nanoscale. This approach leverages the inherent programmability of DNA to create complex nanostructures that can be used as conductive pathways in molecular circuits. In some embodiments, the molecular wire 140 can include a DNA origami structure. The DNA origami can have a predetermined sequence such that the DNA origami organizes into one or more tube or rod structures configured to be disposed across the gap between the electrodes 110 (e.g., a pair of electrodes separated apart by a distance). In some embodiments, the gap or distance between the electrodes 110 can be about 5-50 nm, about 10-20 nm, about 20-40 nm, about 40-100 nm, inclusive of all values and subranges therebetween.

In some embodiments, the tubes or rods of DNA molecules can be held together by “staples” of DNA. As used herein, “staple” refers to a portion of a molecule of DNA configured to hold the DNA origami scaffold structures together. In some embodiments, the DNA origami can provide a plurality of flow pathways for electrons between the electrodes 110 (e.g., a first electrode and a second electrode), thereby increasing conductivity between the electrodes. In some embodiments, the DNA origami may provide more locations at which functional groups can be coupled to the molecular wire 140, as described in further detail below.

In some embodiments, the duplex (or the double stranded portion(s) of the at least one DNA molecule) may be modified to change the conductivity of the molecular wire 140. In some embodiments, the modification of the duplex can include replacing nucleotides. For example, one or more nucleotides may be replaced with molecular structures configured to increase conductivity of the duplex. For example, the one or more nucleotides may be replaced with perylene-3,4,9,10-tetracarboxylic diimides (PTCDIs), which facilitate easy incorporation into the DNA duplex and enhances conductivity through the molecular wire. In some embodiments, modifying the duplex can enhance conductivity (e.g. about 2x to about 10x enhanced conductivity). In some embodiments, an unmodified DNA wire may have a resistance of about 150 to about 200 GigaOhms. In some embodiments, a modified DNA wire may have a resistance of about 10 to about 50 GigaOhms. In some embodiments, the molecular wire 140 (e.g., the DNA wire) may be modified to conduct a level of current sufficient for sensing applications.

Alternatively or additionally, the duplex may be coated with conductive nanoparticles, such as gold nanoparticles, to increase conductivity of the duplex. However, this approach may be cumbersome and variable, as there is no deterministic association between the placement of each conductive nanoparticle with the bases in the nucleic acid backbone, which may cause variable electrical properties.

In some embodiments, modifying the electrical properties of the molecular wire 140 may include replacing the natural nucleotides, or a subset thereof, with modified versions of those bases. The modifications can include coupling functional groups (e.g., functional moieties, conductive units, ligands, etc.) 170 to the individual nucleotides and/or integrating the functional moieties 170 into the individual nucleotides. In some embodiments, functional groups 170 can refer to molecules that are reactive. In some embodiments, functional groups 170 can refer to molecules that are inert. In some embodiments, integration of functional moieties 170 into nucleotides can be accomplished through a substitution, or through modifications to the phosphate backbone. In some embodiments, nucleobase substitutions and/or substitution of 2′-OH may be a more desirable approach when forming duplex-based molecular wires and/or when forming junctions along the molecular wire 140 with three or more oligonucleotides.

In some embodiments, the functional moieties 170 may be conductive and/or enhance conductivity through at least a portion of the molecular wire 140. One important consideration of modification of nucleotides is that the modification(s) does not interfere with hydrogen bonding of the single stranded polymer to its complementary strand. Integration of the functional moiety 170 can be accomplished through the substitution of the functional moiety 170 with one of the native atoms or molecular arms of the nucleotides. The substitution can be implemented on the sugar or the base of the nucleotide. In some embodiments it may be advantageous in some circumstances to have the substitution take place at more than one location on a nucleotide. In some embodiments, the functional moiety 170 may be integrated into the nucleotide via a larger substituent (e.g., a streptavidin), and the larger substituent can bind to a biotin, which has been functionalized with the moiety.

In some embodiments, the functional moieties 170 may include one or more ring structures. In some embodiments, the functional moieties 170 can include large aromatic rings that exhibit highly delocalized electronic configurations and can facilitate migration of electrons or holes. As a result, these functional moieties 170 may enhance the electrical conductivity, thereby reducing the resistivity of the molecular wire 140. The electron orbitals may be pi-conjugated to support electron delocalization (i.e., sharing of electrons between adjacent molecules). In some embodiments, the functional moieties 170 can include pi-conjugation throughout, or in a locally decentralized region, of each modified nucleotide. For example, the functional moieties 170 can cause sharing of electrons between different molecular groups throughout the modified nucleotide and/or at certain portions of the modified nucleotide. The electron delocalization can facilitate efficient electron transfer through the modified nucleotide (and therefore through the molecular wire 140).

In some embodiments, the functional moieties 170 can include redox reporters. In some embodiments, the redox reporters can be coupled to the nucleotides and/or the double stranded backbone. In some embodiments, the redox reporters can include methylene blue, for example.

In some embodiments, a spacing between modified nucleotides in the single stranded oligo used in the molecular wire can be experimentally optimized (e.g., for a particular application). In other words, the functional groups 170 can be coupled to nucleotides at predetermined positions along the DNA strand(s) of the molecular wire 140. It is expected that the conductivity of the resulting molecular wire can increase with an increased frequency of nucleotides modified with conductive functional moieties 170. In some embodiments, every base used in the molecular wire 140 may be replaced with a modified analogue, which would support significant interaction between the electronic orbitals of adjacent nucleotides in the at least one molecule of DNA of the molecular wire 140. When there is pi-orbital overlap between adjacent, or neighboring nucleotides, the orbital interaction can facilitate a delocalization of the electrons in the molecular wire 140, thereby enhancing conductivity (e.g., by reducing resistivity) through the molecular wire 140 and between the electrodes 110 of the device 100. It is also expected that at a certain separation between modified nucleotides (e.g., a specific number of bases), the interaction of the adjacent functional moieties is zero or near zero, and the conductivity of the molecular wire 140 may return to that of the unmodified duplex.

Therefore, in some embodiments, nucleotides modified with functional moieties 170 may have a minimum spacing there between such that pi-orbitals overlap between adjacent functional moieties 170. Through deterministic placement of modified nucleotides and non-modified (natural) nucleotides, and through the selection of the functional moiety(ies) 170, the electrical properties of the resulting molecular wire 140 can be deterministically engineered. In other words, molecular wires 140 described herein may include modified nucleotides and non-modified (natural) nucleotides located at predetermined locations along a length of the molecular wire 140 to cause the molecular wire to have predetermined electrical properties such as increased electrical conductivity. In some embodiments, the functional moieties 170 can be spaced every 1, every 2, every 3, every 4, every 5, every 6, every 7, every 8, every 9, every 10, every 20, every 30 nucleotides, etc. In some embodiments, the functional moieties 170 can be spaced on nucleotides of a particular orientation with the double helix twist (e.g., such that the functional moieties 170 are substantially aligned along a plane). In some embodiments, the functional moieties 170 may be spaced along the molecular wire in a repetitive orientation (e.g., to facilitate pi-orbital overlap and electron delocalization).

In some embodiments, the molecular wire 140 may include functional groups 170 that allow the molecular wire 140 to function in a manner that is analogous to that of a conventional conductor, semiconductor, and/or insulator. Introducing functional moieties 170 on the molecular wire 140 can have a similar effect as introduction of dopants, through methods such as ion implantation, that facilitate the tuning of the electrical properties of bulk materials.

A double stranded duplex may be formed from a single stranded oligo that includes functional moieties 170. In some embodiments, the complementary strand may also include modifications. In some embodiments, the complementary strand may not include functional moieties 170. In some embodiments, one or both single stranded oligos of the double stranded duplex of the molecular wire 140 may include functional moieties 170. In some embodiments, the inclusion of functional moieties 170 on a single stranded oligo and its complementary strand may enable the desired performance (e.g., level of conductivity).

By controlling the physical properties and the spacing (frequency) of the functional moieties 170 throughout the oligonucleotide polymer of the molecular wire 140, properties of the molecular wire 140 (e.g., conductivity) can be deterministically controlled. For example, adding more functional groups 170 may facilitate increased or decreased electrical conductivity. The physical properties of the functional groups 170 can include electron donating groups, electron withdrawing groups, hydrophobic groups, hydrophilic groups, charged groups, polarizable groups, and the like. In some embodiments, the functional groups 170 may be configured to be switched on/off (activated) through optical, electrical or chemical methods (i.e., their properties may change upon exposure to noted conditions).

In some embodiments, the functional groups 170 may include metal atoms, aromatic moieties (both with and without metal atoms incorporated into the molecular structure), linear molecules, redox reporters, etc. The functional moieties 170 added to the nucleotides may be determined based on desired physical properties of the molecular wire 140, such as the ability to donate or accept electrons (electron affinity), the local surface energy of the molecular wire 140 (hydrophobicity, hydrophilicity, etc.), the electrical conductivity (or inversely, the resistivity), the specific heat of the molecular structure (e.g., melting temperature of hybridized duplexes) and/or the ability to interact with specific target molecules that may be probed by the sensor.

The functional moieties 170 may cover a broad range of molecular species, including phenyl, naphthyl, vinyl, tryptamino, tryptophan, isobutyl, epoxy, analine groups, and similar molecular structures. In some embodiments, a branched ligand (e.g., a functional moiety) may be integrated, which may include a plurality of functional groups 170 (e.g., similar functional groups or different functional groups) on a single nucleotide. In some embodiments, the molecular wire 140 may include one of these functional moieties 170 integrated into a nucleotide and then the modified nucleotide may be integrated into an oligonucleotide strand of the molecular wire 140 to control the properties of the molecular wire 140. Furthermore, addition/integration of electron rich atoms, such a Fe, Cu, Ni, Pd, Ag, Au, Pt, Cr, Ti, are potentially advantageous due to their ability to readily donate and accept electrons (they are intrinsically conductive). Increasing a number modified nucleotides in the oligonucleotide with functional groups 170 that donate electrons (or encourage electron delocalization) can increase conductivity and/or decrease resistivity of the molecular wire 140. The inclusion of a metal atom can influence (e.g., increase) the conductivity relative to use of the non-substituted group.

In some embodiments, the functional moieties 170 (or functional groups) can include large aromatic rings with a metal atom in the center. These structures can form orthogonal pi orbitals in which the metal atom can act as a donor of electrons. Example modifications may include addition of a phenyl group onto a nucleotide or addition of an iron-based molecule including heme groups such as ferrocene, etc. In some embodiments, the molecular wire 140 can include the same type of functional moieties 170 across the molecular wire 140. In some embodiments, the molecular wire can include different types of functional moieties 170 across the molecular wire 140. In some embodiments, the molecular wire can include a combination of functional moieties 170 that cooperate with one another to accomplish a particular function.

Alternatively, substituting functional group(s) or an atom, or a collection of atoms, that have an elevated electron affinity (e.g., strong acceptors of electrons) may reduce the likelihood of electron mobility through the wire (increase resistivity).

In some embodiments, molecular wires 140 described herein may have a reduced level of conductivity to create a molecular insulator. In some embodiments, the molecular wire 140 may be configured as a semiconducting molecular wire 140. For example, at least a portion of the molecular wire 140 may be configured as an insulator such that the circuit is a closed circuit that does not conduct electrons through the molecular wire 140 until a sufficient voltage is applied, after which (for voltages greater than a threshold voltage) a current flow through the molecular wire 140 is enabled or occurs. In some embodiments, the molecular wire 140 may be configured to operate as a semi-conductor.

The functional moieties 170 integrated with the nucleotides may also be nanostructures themselves. These nanostructures may be constructed out of small molecules such as nucleic acids, polymers, or a combination thereof. For example, a nucleic acid-based moiety may include DNA origami or nucleic acid nanostructures such as hairpins or secondary structures periodically disposed or arranged (e.g., disposed at predetermined intervals) along the oligo. In some embodiments, these secondary structures may be configured to provide additional functionality (in addition to impacting electrical properties) such as, for example: (i) performing logic operations, (ii) gating where electrical performance is only activated in the presence of complementary strands, or (iii) other complex operations. In some embodiments, these logic operations and/or gating properties can be catalyzed through the introduction of additional short oligos.

In some embodiments, the device 100 can include one or more transduction agents 180 (e.g., proteins such as enzymes, genetic probes, anti-bodies, and/or affinity binders, aptamers, etc.) configured to be coupled to the molecular wire(s) 140. In some embodiments, the integration of a protein or a collection of proteins with or into the molecular wire assembly can enable manipulation and/or modulation of the current flowing through the molecular wire 140. In some embodiments, the transduction agent 180 can include a protein configured to change conformation in response to binding to a target structure (e.g., a nucleotide, a sequence of DNA, a protein, or other nanostructure). In some embodiments, the change in conformation of the transduction agent 180 can change electrical properties such as conductivity through the molecular wire 140. In this way, molecular events, such as changes in structure in protein(s) integrated into the DNA wire, can be determined based on changes in performance parameters of the molecular wire 140.

For example, the current or voltage measured between the electrodes 110 can change in response to the transduction agent 180 performing undergoing one or more conformational changes. A change in performance of the wire 140 can include, for example, a change in current flowing through the circuit and serves as the basis of the observable property of the sensor. Through precise and controlled engineering of the molecular wires 140, or engineering of biomolecules such as proteins that are linked to the molecular wire, the integration of functional groups 170, and the inclusion of a transduction agent 180 such as a protein, a single strand of DNA, or the like, the properties of the sensors can be controlled and utilized for specific applications.

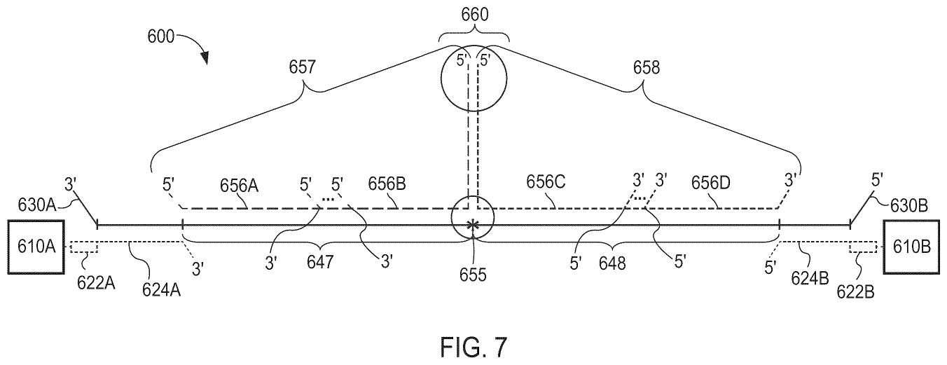

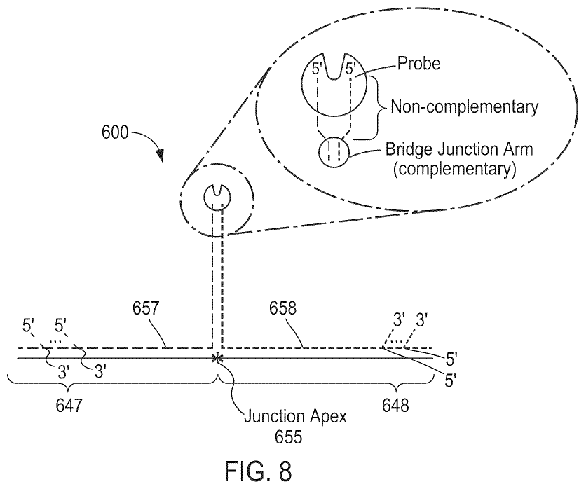

The integration of functionality into the molecular wire 140, such as adding branches to the circuit or adding proteins such as a polymerase or affinity binders (e.g., antibody(ies), aptamer(s), peptide(s), ssDNA, any other affinity binders, or any suitable combination thereof) can enable transduction of the presence of a target molecule in an environment surrounding the device 100 into a detectable signal measured between electrode(s) 110. For example, a polymerase can be integrated into the molecular wire 140, as described in FIGS. 7-8. Through measuring the current flowing through the molecular wire 140, the activity of the polymerase can be monitored.

Examples of polymerases include Taq DNA polymerase and/or Phi 29 DNA polymerase, or engineered/mutated versions of these enzymes. Through the conjugation of ssDNA into specific locations within the polymerase of interest, one can enable incorporation of the polymerase into the molecular wire 140, which then enables (a) monitoring of whether incorporation of a base into an oligo being probed has occurred or not, (b) monitoring which base was incorporated, and/or (c) determining which nucleotide was attempted to be incorporated but failed, all of which result in the ability to directly read the process of converting single stranded DNA into double stranded DNA and effectively reading the sequence of the nucleotides as this process takes place. In other words, a transduction agent 180 such as a polymerase can enable the device 100 to sequence target strands of DNA. In some embodiments, site specific conjugation can be used to bind the transduction agent 180 to the molecular circuit. In some embodiments, a polymerase can be surveyed for a pair of conjugation areas that do not interfere with the polymerase ability to process DNA, and the molecular wire 140 can be attached to the polymerase at the pair of conjugation areas. In other words, the polymerase can include a binding site separate from an active portion of the polymerase. The binding site can be configured to bind to the molecular wire 140.

In some embodiments, the one or more functional groups 170 can be coupled to catalytic operators such as polymerases to enhance electrical properties (e.g., conductivity) of the transduction catalytic operator. In some embodiments, the incorporation of the one or more functional groups 170 can be site specific. In some embodiments, the incorporation of the one or more functional groups 170 can be random. In some embodiments, the one or more functional groups 170 can be configured to modulate current flowing through the catalytic operator in a way that is correlated with the processivity of the catalytic operator. Therefore, the one or more functional groups 170 can facilitate modulation of current flowing between the electrodes 110 of the circuit based on the catalytic operator activity.

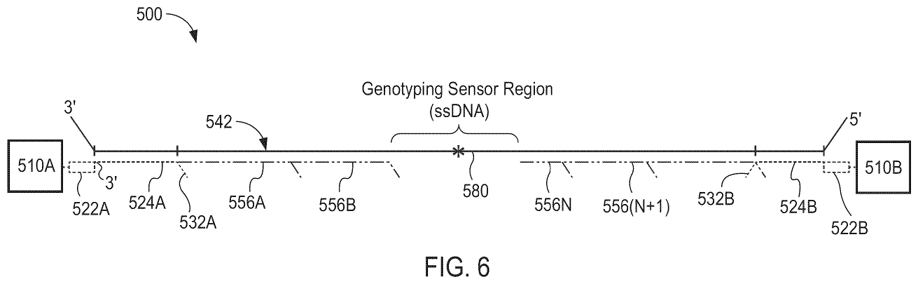

In some embodiments, the molecular wire 140 may include a single stranded DNA section configured to probe solution for a complementary sequence (e.g., a genotyping sensor region), as described in FIG. 6. In some embodiments, a current through a probe region when the probe region is in the single stranded state can be altered (e.g., turned on and/or off) by the formation of the duplex (e.g., the double stranded state). In some embodiments, the molecular wire 140 may include a bridge (e.g., including at least a portion of dsDNA) connecting two electrode terminals 110. The bridge may include a central zone. In some embodiments, the central zone may include dsDNA. In some embodiments, a series of bases of the duplex can be removed such that a single stranded zone (e.g., the probe region) is formed. In some embodiments, the sequence of the ssDNA zone may be configured to probe a predetermined complementary sequence. When the complement to the probe is not present, a first level of current may be measured through the molecular wire 140. When the complement to the probe is present, the molecular wire 140 can transition to a double stranded state (e.g., the entire wire is double stranded), and a second level of current can be measured, the second level of current being larger than the first level of current.

In some embodiments, the transduction agent 180 can include an affinity binder configured to be integrated into the molecular wire 140 and to monitor the current flowing through the molecular wire 140 when the molecular wire 140 is exposed to a target sample. For example, the device 100 may be configured to determine an identity of one or more proteins present in a sample and a concentration of respective proteins using the molecular wire(s) 140. As with the polymerase, the affinity binder probes can interact with various molecules, and the current flowing through the molecular wire 140 may change depending on the interaction of that affinity binder with the target molecules. The affinity binder binding to an epitope with which it has a strong interaction with (i.e. the binder finds the protein for which it was designed to bind to) can result in a change in the current going through the molecular wire 140, thereby resulting in a measurable signal associated with the binding event. In some embodiments, the measurements may be collected in real-time or near real-time, for example, when the polymerase or any other sensor molecule coupled to the molecule wires 140 described herein performs its intention operation (e.g., a natural operation such as catalysis, synthesis, etc.) or a modified version of its intended operation. The real-time monitoring of the binding and then dissociation of an affinity binder with a collection of target molecules can give detailed information about the molecules being surveyed. For example, when the affinity binder binds with a molecule, the duration of the interaction can be measured through monitoring the duration for which a change in current through the circuit is observed, thus giving a direct measurement of the kinetics of the interaction between the affinity binder and the molecule it is interacting with.

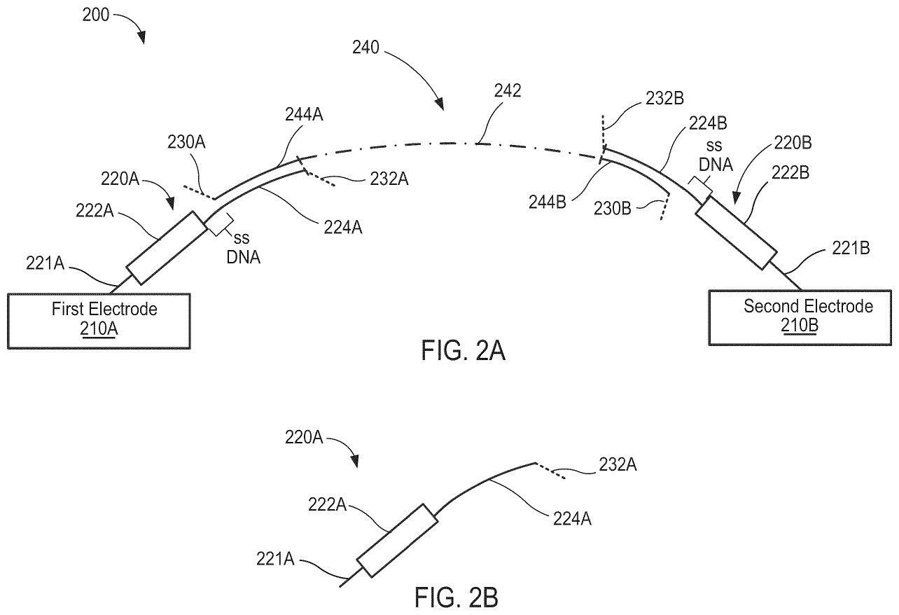

In some embodiments, the molecular wire 140 can include one or more toehold regions or toehold sequences (e.g., sequences 230A, 230B, 232A, 232B shown in FIG. 2A) configured to facilitate toehold mediated strand displacement actions. In some embodiments, the toehold sequences can enable surface regeneration of at least a portion of a DNA strand in the molecular wire 140 that includes the toehold sequence. In some embodiments, the molecular wire 140 can include a first toehold region and a second toehold region flanking a portion of a DNA strand in the molecular wire 140 such that the portion of the DNA strand can be replaced via toehold mediated strand displacement.

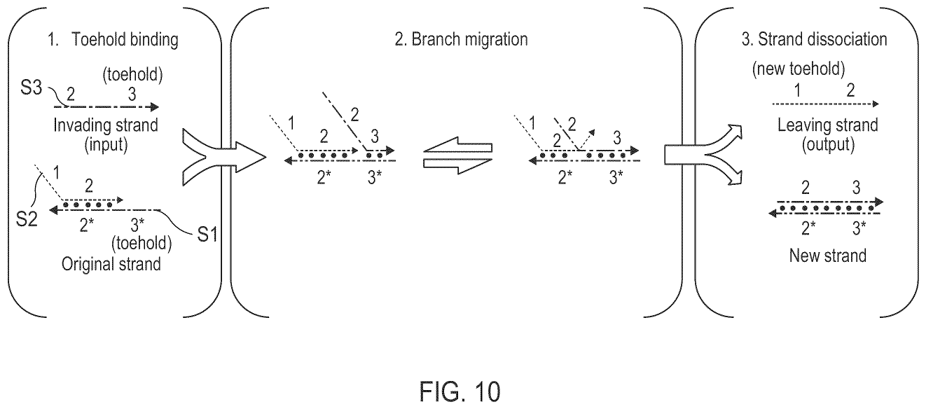

In some embodiments, a first terminal end of the molecular wire 140 can include a first toehold region and a second terminal end of the molecular wire 140 can include a second toehold region. The toehold regions on both terminal ends can support toehold mediated strand displacement such that the molecular wire 140 can be replaced with a new molecular wire 140, for example, to refresh or renew the device 100. In some embodiments, the molecular wire 140 can be replaced with a molecular wire having the same DNA sequence, functional groups, and/or function. In some embodiments, the molecular wire 140 can be replaced with a molecular wire having a different DNA sequence, functional groups, and/or function. In some embodiments, the molecular wire 140 can include a plurality of toehold regions such that one or more portions of the molecular wire 140 can be replaced. For example, the molecular wire 140 can be modular, meaning the molecular wire 140 is formed of a plurality of oligos including toehold regions at each terminal end of the respective oligo. Therefore, one or more of the plurality of oligos can be replaced via toehold strand displacement. In some embodiments, certain portions of the molecular wire 140 may not include toehold regions such that toehold displacement cannot occur at that portion of the molecular wire 140. The process of toehold mediated strand displacement is described in further detail with respect to FIG. 10. The one or more toeholds can allow sensor regeneration and/or sensor reprogramming by replacing or modifying at least a portion of the molecular wire 140.

In some embodiments, the one or more linkers 120 can be configured to attach a portion of the molecular wire 140 to a predetermined electrode 110. In some embodiments, the one or more linkers 120 can be configured to attach the portion of the molecular wire 140 to a predetermined location on a predetermined electrode 110 of an electrode array (e.g., on the semiconductor 105). In some embodiments, the linker(s) 120 can include a first linker disposed on a surface of the electrode 110 and a second linker attached to the portion of the molecular wire 140. The first linker and the second linker may be configured to bind to one another to couple (e.g., attach or anchor) the molecular wire 140 to the electrode 110.

The one or more linkers 120 can be molecular linkers including one or more linker molecules. The linker molecules may covalently bind to the electrodes (e.g., via covalent bonds), and provide a handle to which the molecular wire 140 can be coupled, and/or from which the molecular wire 140 can be constructed. Example chemistries for linking the molecular wire 140 to the electrodes 110 may include thiol chemistry (e.g., with electrodes containing gold), click chemistry, and/or metal-carbon (e.g., Au—C) covalent linkages, as described in further detail below with respect to FIGS. 2A-2B.

Conventionally, loading single molecules into arrays is done without the ability to precisely control the location and the ability to deterministically place the molecules is governed by statistical events (i.e., Poisson statistics). The upper limit on control with these processes is that ˜33% of molecules will be positioned where they are intended to be placed. In other words, loading of the molecules generally follows Poisson loading. In some embodiments, the device 100 may employ DNA origami to overcome Poisson loading, for example, allow Super Poisson loading of molecules on a surface. For example, in some embodiments, the linkers can include a DNA origami. For example, a DNA origami can be configured to bind to a surface of the electrode(s) 110. The DNA origami can be engineered to be size-matched to the end of the electrode 110 and provide several covalent linkage sites for binding molecules (e.g., the toe-hold regions) to the electrode(s) 110 (e.g., the first electrode and the second electrode). This linking technique can be an efficient way to conduct electricity from the first and second electrodes to the molecular wire 140. Using DNA origami as linkers can enable use of larger electrodes 110. In some embodiments, the electrodes can be about a factor of 2 larger than the molecular wire 140.

In some embodiments, the DNA origami can include a plurality of staples including programmable sites (also referred to as “sites”). The programmable sites can include a predetermined sequence of DNA that is programmable. For example, the DNA origami can include a plurality of scaffold structures and a plurality of staples at the junction point(s) of the scaffold structures to connect the scaffold structures. The staples include molecules of DNA configured to hold the DNA origami scaffold structures together, and the programmable site refers to a portion of the staple configured to hybridize to the molecular wire 140. In some embodiments, the staple can include a predetermined sequence that is complementary to a respective portion of the molecular wire 140. Therefore, the DNA origami can provide more sites at which the molecular wire 140 can bind to the electrodes 110. In some embodiments, the DNA origami can enable binding of a plurality of molecular wires 140 to a respective electrode 110. In some embodiments, the programmable sites (or portions of the DNA origami near the programmable sites) can include toehold regions such that the programmable sites can be replaced and/or reprogrammed via toehold mediated strand displacement. In some embodiments, at least a subset of the staples are unique (e.g., having a unique DNA sequence and/or structure).

With the use of DNA origami, super-Poisson loading (i.e., higher loading than expected in a Poisson distribution or loading with a probability distribution that has a larger variance than a Poisson distribution with the same mean) of molecules into desired locations can be achieved and leveraged to create complex patterns with nanoscale precision. DNA origami can be integrated on flat or microstructured surfaces (e.g., micro or nanotextured surfaces). Directed self-assembly is another technique that can be used to create supra-molecular patterns from smaller building blocks. DNA origami can also be used as a “molecular breadboard” such that the molecular wire 140 can electrically connect to the electrode terminal at any of the binding sites, or a subset of the binding sites of the DNA origami. Typical dimensions of origami particles may be about 100 nm×100 nm, with a molecular breadboard (e.g., binding sites) superimposed on these particles having a resolution of about 3 nm to about 5 nm, inclusive. Through integration of target molecules with DNA origami, one has the ability to place single molecules with extreme accuracy and precision.

The interface between the metallic electrodes 110 and the molecular wire 140 can be a junction of high resistivity. In some embodiments, electron transfer can be mediated by tunneling. For example, a gap or space between the molecular wire 140 and the electrode interface of the electrodes 110 can be sufficiently small and/or certain functional groups can be disposed near the interface such that the gap is tunnelable. In some embodiments, the molecular wire 140 can include a DNA origami decorated with functional groups (e.g., a polymerase), and tunneling of electrons through the gap between the electrodes can be measured.

In some embodiments, to improve conductivity at the electrode 110 and molecular wire 140 interface, one or more portions of the linker 120 (e.g., DNA origami linker, a ssDNA linker, and/or any other linker described herein) can include one or more functional groups 170 coupled thereto configured to increase conductivity through the linker 120, and therefore increase conductivity between the electrode 110 and molecular wire 140 interface. In some embodiments, a single face or both faces of the DNA origami can the one or more functional groups 170. In some embodiments, the linker 120 can include a plurality of functional groups 170 configured to promote merging of electronic structures. For example, the highest occupied molecular orbital (HOMO) of the functional group can be similar energy to the Fermi level of the electrode such that electron transfer is promoted between the electrodes 110 and the functional group. In some embodiments, the DNA origami can be coupled to a plurality of functional groups 170 including large aromatic rings such that the pi orbitals of the large aromatic rings extend toward and away from the surface of the electrodes 110 (e.g., orthogonal or substantially orthogonally). This orientation of the pi orbitals may allow for strong coupling between electrons in the material of the electrodes 110 and the DNA molecules of the DNA origami. In some embodiments, the functional groups 170 can be coupled to the programmable sites or staples (e.g., where the molecular wire 140 is configured to bind) to increase electron transfer where the molecular wire 140 binds. In some embodiments, the functional groups 170 of the DNA origami linker can include pyrrole, heme groups, or any other aromatic ring described herein, or any suitable combination thereof.

In some embodiments, electrode surface of the electrodes 110 and/or the linker can be configured to promote thermionic emission or thermionic sharing of electrons. For example, a thermal energy of the electrode surface can be controlled to promote electron transfer to the linker 120. In some embodiments, impedance at the electrode surface can be controlled based on electronics in the semiconductor chip 105. For example, the semiconductor chip 105 can include a gating mechanism (e.g., such as a transistor) patterned into the semiconductor chip 105 under the electrode(s) 110) to modulate an electron or hole concentration, and therefore, control conductivity of the semiconductor material. In some embodiments, the gating mechanism can be used to accumulate electrons at the one or more of the electrodes 110. In some embodiments, the gating mechanism can be a solution-based gate.

In some embodiments, the electrode(s) 110 can disposed on and/or patterned into the semiconductor 105 (e.g., a semiconductor chip 105), and the device 100 can include a plurality of molecular wires 140. In some embodiments, each molecular wire 140 of the plurality of molecular wires configured to electrically connect a set of electrode terminals of the plurality of electrodes 110 to form one or more molecular circuits. In some embodiments, the semiconductor chip 105 can be a pre-fabricated chip such as a CMOS chip. In some embodiments, the semiconductor chip 105 can be programmed with circuitry to control electrical signals between one or more electrodes 110 of the device 100. In some embodiments, the semiconductor chip 105 can include circuitry configured to apply voltage between subsets of the electrodes 110, read current levels between subsets of the electrodes 110, and/or include one or more gating or switching mechanisms.

In some embodiments, the device 100 can be configured as a biological based programmable sensing array. For example, the semiconductor chip 105 can include a plurality of electrodes 110 electrically connected by a plurality of molecular wires 140, and the plurality of molecular wires 140 can be configured to perform a programmed function. In some embodiments, a single unit cell can consist of several closely packed (within 100×100 nm² ) nanogap electrodes 110, deterministically interconnected by double-stranded DNA-based (dsDNA) bridges (e.g., nanowires) 140 that can be branched to create a biomolecular circuit network.

In some embodiments, the biological based programmable sensing array can be analogous to a Field Programmable Gate Array (FPGA) that enables a user to reconfigure hardware to meet specific use case requirements. For example, the device 100 can include molecular architectures that can be user defined and created in real-time based on electrically directed nanoscale control of transduction agents 180 (e.g., single molecular catalysts). The device 100 can enable customization through introduction of a user defined set of reagents. This reagent set can be formulated upon a base molecular foundation, leveraging biomolecules such as nucleic acid polymers and enzymes, that can enable integration into the hardware circuitry.

In some embodiments, the device 100 can include an Application-Specific Integrated Circuit (ASIC) implemented in a CMOS (e.g., 55-nm) technology having an array of individually addressable control pixels, each with a low-leakage multiplexer (Mux) and local registers for electrode selection. Selected electrodes 110 can connect to either readout channels and/or a programmable voltage source. In some embodiments, the readout channels may use a low-noise, high-sensitivity transimpedance amplifier and an analog-to-digital converter (ADC). In some embodiments, a digital controller may manage pixel programming and overall chip functionality. In some embodiments, the device 100 can have molecular circuits or sensors a distributed with a pitch of about 200 nm to about 500 nm, inclusive of all values and ranges therebetween.

In some embodiments, the device 100 can include a plurality of transduction agents 180 (e.g., catalysts or catalytic operators). In some embodiments, the catalysts or catalytic operator can be organized in such a way that a collective suite of operations (e.g., enzyme cascades), such as a multi-step chemical synthesis process, can be performed. In some embodiments, each catalytic operator may be configured to utilize a product or biproduct of a reaction of the previous catalytic operator. In some embodiments, the plurality of transduction agents 180 can be configured to work in parallel and/or to work in coordination to perform sensing operations, sequencing operations, and/or perform any suitable chemical reaction or series of chemical reactions. In some embodiments, the catalytic operators may be localized based on a programmable sequence of the molecular wire 140 (e.g., the first binding region and the second binding region) and programmable sequence(s) of the linker(s) 120 (e.g., the DNA origami and/or foundation oligos described in detail below).

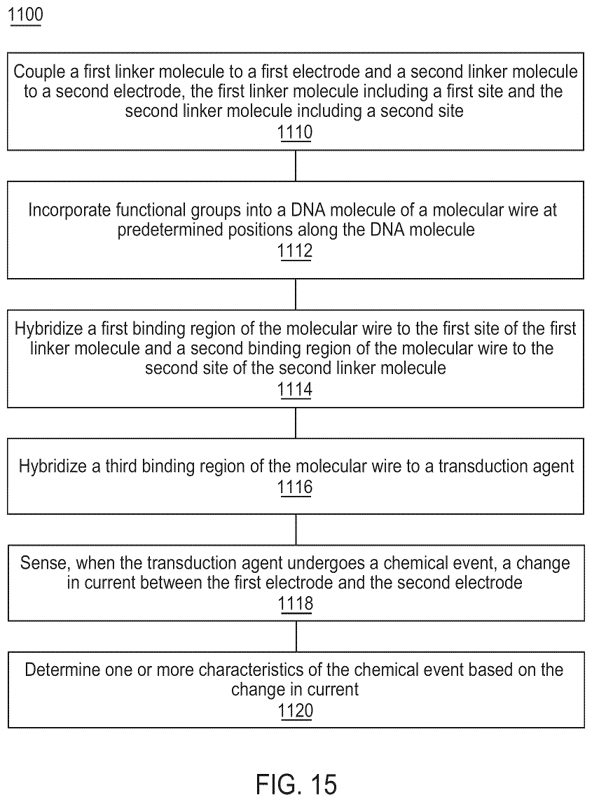

After successfully optimizing the protocols for placement of individual catalysts ˜100 sites, embodiments can include dedicated ASIC to extend this to >1,000 sites. Control of an enzymatic cascade (containing at least 6 unique catalysts) can be achieved by branching of the DNA nanowire, with each branch include a unique enzyme binding site (e.g., as shown in FIG. 15). This approach makes use of networked DNA nanowires, electrically connected and tethered with nanoelectrode terminals to a controlling CMOS-based ASIC, to mount, control, and sense any arbitrary cascade configuration of (designed) enzymes. The purpose of the DNA nanowires 140 is two-fold: (1) through highly sequence-specific hybridization, they provide a bottom-up precision assembly methodology, and (2) their conductive nature guides and facilities electron transport between the solid-state control circuits and the enzymes.

In some embodiments, the transduction agents can include catalysts or catalytic operator, and the catalysts or catalytic operator can be localized in a predetermined portion of the molecular circuit based on a sequence of the molecular wire 140 and the function of the catalyst. In some embodiments, control and/or localization of the catalysts or catalytic operator can be based on: i) redox-control binding/affinity/substrate specificity (e.g., [4Fe-4S] cluster proteins [REF]); ii) catalytic energy transfer (e.g., replacing the need for a co-factor); and/or iii) localized substrate/cofactor (re)generation (e.g., dATP).

In some embodiments, measurements of the molecular wire 140 can be conducted using techniques including, for example, atomic force microscopy (AFM) and scanning tunneling microscopy (STM). These techniques can allow characterization of the structural and electrical properties of the molecular wires, ensuring that they meet the desired specifications for integration into the biological based programmable array.

In some embodiments, the semiconductor chip 105 can be operatively coupled to a processor (not shown) configured to receive one or more signals from the semiconductor chip 105. For example, the processor can be configured to receive (e.g., as the transduction agent 180 sequentially binds to a target molecule) signals corresponding to a level of current directed between the pair of electrodes 110. In some embodiments, the processor can be configured to receive signals as a polymerase binds to nucleotide and changes conformation. In some embodiments, the processor may be configured to determine a sequence of a target strand of DNA based on the signals.

In some embodiments, the processor may be located on an external device and configured to receive one or more signals from the semiconductor 105. The processor can be any suitable processing device(s) configured to run and/or execute a set of instructions or code. For example, the processor can be and/or can include one or more data processors, image processors, graphics processing units (GPU), physics processing units, digital signal processors (DSP), analog signal processors, mixed-signal processors, machine learning processors, deep learning processors, finite state machines (FSM), compression processors (e.g., for data compression to reduce data rate and/or memory requirements), encryption processors (e.g., for secure wireless data and/or power transfer), and/or the like. The processors can be, for example, a general-purpose processor, central processing unit (CPU), edge computing and/or edge AI processor, edge machine learning processor, and/or the like.

FIG. 2A shows a device 200 including a molecular wire 240 (e.g., a DNA based wire) configured to couple a first electrode 210A and a second electrode 210B, according to an embodiment. In some embodiments, the first and second electrodes 210A, 210B may be fabricated with conventional top-down processes and using conventional materials. In some embodiments, the electrodes 210A, 210B may be included in a circuit fabricated using conventional semiconductor grade manufacturing methodologies (e.g., top-down methods). In some embodiments, the first and second electrodes 210A, 210B can be included on a semiconductor chip (not shown). In some embodiments, the first and second electrode 210A, 210B can be coupled to additional circuitry and/or electronics on the semiconductor chip. In some embodiments, the device 200 can be integrated via a bottom-up self-assembly process (e.g., 3-D printing, sputtering, e-beam deposition, thermal deposition, chemical vapor deposition (CVD), plasma enhanced chemical vapor deposition (PECVD), atomic layer deposition (ALD), etc.). In some embodiments, the first electrode 210A and the second electrode 210B may include any suitable electrically conductive material such as, for example, gold, platinum, copper, silver, titanium, conductive polymer, carbon-based, any other suitable conductive material, or any suitable combination thereof.

In some embodiments, the device 200 may further include one or more linker complexes (referred to herein as “linkers”) 220A, 220B, shown enlarged in FIG. 2B. For example, the device 200 may include a first linker molecule 222A and a second linker molecule 222B. In some embodiments, the device 200 may include a plurality of linkers. The first and second linker molecules 222A, 222B may (i) covalently bind to the electrodes 210A, 210B (e.g., via covalent bonds 221A, 221B) and (ii) provide a handle to which the molecular wire (nanostructure) 240 can be coupled, and/or from which the molecular wire 240 can be constructed. In some embodiments, the linkers 220A can include a DNA origami. For example, a DNA origami can be configured to bind to the electrodes 210A, 210B. The origami can be engineered to be size-matched to the end of the electrodes 210A, 210B, and include several covalent linkages to the first and second electrodes 210A, 210B. This linking technique can be an efficient way to conduct electricity from the first and second electrodes 210A, 210B to the molecular wire 240. In some embodiments, DNA origami may be used to overcome Poisson loading (e.g., to provide Super-Poisson loading). In some embodiments, the DNA origami can include a plurality of programmable sites having a predetermined sequence that is complementary to a respective portion of the molecular wire 240. Therefore, the DNA origami can provide more sites at which the molecular wire 240 can bind to the electrodes 210A, 210B. In some embodiments, the DNA origami can enable binding of a plurality of molecular wires 240 to a respective electrode 210A, 210B.

The device 200 may further include a bridge oligo 242 which couples, connects, or links the two linkers 220A, 220B, and therefore couples (e.g., electrically connects) the two electrodes 210A, 210B to each other. In some embodiments, each of the handle molecules 224A, 224B on the linkers 222A, 222B, and the bridge oligo 242 can include DNA. In some embodiments, the DNA may include toehold regions 230A, 230B, 232A, 232B. The toehold regions 230A, 230B, 232A, 232B may enable surface regeneration for at least a portion of a DNA strand including a toehold region 230A, 230B, 232A, 232B. In some embodiments, a portion of the molecular wire 240 may not include toehold regions 230A, 230B, 232A, 232B such that surface regeneration is not possible at the portion of the molecular wire 240 that does not include the toehold region 230A, 230B, 232A, 232B. The DNA may, or may not be, modified, as described herein.

In some embodiments, the linkers 220A, 220B device 200 may include foundation oligos (FO) 224A, 224B immobilized on the one or more electrodes 210A, 210B. FIG. 2B shows an enlarged view of a foundation oligo 224A. In some embodiments the foundation oligo strands 224A, 224B may each be formed of ssDNA. In some embodiments, a foundation oligo 224A, 224B may be covalently bonded to an electrode 210A, 210B and include ssDNA that is configured to hybridize to the bridge oligo 242 such that no unpaired bases exist (e.g., the foundation oligo 224A, 224B has no single strand portion when hybridized to the bridge oligo 242). As shown, the first linker molecule 222A includes a first foundation oligo 224A attached thereto and the second linker molecule 222B includes a second foundation oligo 224B attached thereto. The first foundation oligo 224A and the second foundation oligo 224B can include a predetermined sequence of a predetermined length that is complementary to a respective portion of the bridge oligo 242 to connect the bridge oligo 242 to the electrodes 210A, 210B.

In some embodiments, the foundation oligo 224A, 224B may include a foundation toehold region 232A, 232B located at a terminal end of the foundation oligo 224A, 224B that is not bonded to the electrode 210A, 210B.

In some embodiments, the sequences for each foundation oligo 224A, 224B can be the same. In some embodiments, the sequence for each foundation oligo 224A, 224B can be different from each other. In some embodiments, each of the foundation oligos bound to the same electrode surface are the same. In some embodiments, whether the sequences are the same or different may depend on at least one of a function of the circuit, a desired conductivity of the wire, one or more functional groups coupled to the wire, a relative position of the electrodes, etc. The sequence of the first foundation oligo 224A does not have to be the same as the sequence of the second foundation oligo 224B, and in some instances there are advantages for both foundation oligos 224A, 224B having the same sequence, and other instances where it is advantageous for the two sequences to be different. In some embodiments, including different sequences on each of the electrodes 210A, 210B may provide a marker, indicator, or identifier for a specific electrode or electrode pair. For example, different sequences on each of the electrodes may provide information related to directionality (e.g., which end of the circuit an operation such as strand displacement is being). In some embodiments, including different sequences on different electrodes 210A, 210B may also enable a decoding operation and provide information related to what sequences (e.g., for genotyping applications) are on which electrode pair. In some embodiments, a total number of foundation oligos 224A, 224B in the device 200 can be equivalent to a total number of electrodes 210A, 210B. While FIG. 2A shows two electrodes 210A, 210B and two foundation oligos 224A, 224B, it should be appreciated that the device 100 may include any suitable number of electrodes and/or foundation oligos. For example, the device 100 may include between 1 electrode and 5 electrodes, or an even higher number of electrodes. In some embodiments, any number of molecular wires 240 can be coupled to any combination of the electrodes 210A, 210B.

As shown, the device 200 includes the first foundation oligo 224A coupled to the first electrode 210A via the first linker 222A and the second foundation oligo 224B coupled to the second electrode 210B via the second linker 222B. Each of the foundation oligos 224A, 224B may include a first end and a second end. The first end may be coupled to a respective electrode 210A, 210B via the linker 222A, 222B. In some embodiments, the linkers 220A, 220B may serve as electrical conduits configured to pass current from the electrodes 210A, 210B to the DNA wire 240 and vice versa. In some embodiments, the linkers 220A, 220B may be configured as or include functional elements (e.g., switches, amplifiers, etc.). Each foundation oligo 224a, 224b includes a set of bases (e.g., terminal bases) which form a toehold 232A, 232B on the second end of the foundation oligo 224a, 224b (e.g., on an end portion furthest from the corresponding electrode 210A, 210B). In some embodiments, each foundation oligo 224A, 224B may be directly bound to the electrodes 210A, 210B without linkers 222A, 222B. In some embodiments, the binding of each foundation oligo 224a, 224b to each of the electrodes 210A, 210B may be covalent (e.g., via the bonds labeled 221A, 22B in FIG. 2A). In some embodiments, binding between the first end of the foundation oligo(s) 232A, 232B and the electrode(s) 210A, 210B is enabled through a molecular modification of the first end of each of the foundation oligo(s) 224A, 224B. For example, the 3′ end of 224A may be modified and the 5′ end of 224B may be modified. The binding chemistry and/or molecular modification may be determined based on a desired distance between the foundation oligo 224A. 224B and the electrode surface. In some embodiments, the binding chemistry and/or molecular modification may minimize or reduce a distance between the first end of the foundation oligo(s) 224A, 224B and the surface of the electrode(s) 210A. 210B. In some embodiments, the binding chemistry and/or molecular modification may result in the first end of the foundation oligo(s) 224A, 224B being a predetermined distance from the surface of the electrodes 210A, 210B. The binding chemistry between the foundation oligo(s) 224A, 224B and the electrode interface 210A, 210B may enable sufficient electron transfer between the electrodes 210A, 210B and the molecular wire 240. For example, a molecule, or collection of molecules, (e.g., linkers 222A, 222B) that facilitate the binding of the foundation oligo 224A, 224B to the electrodes 210A, 210B (i.e. the coupling chemistry) may have an electronic orbital structure that facilitates sufficient/efficient electron transfer between the electrode metal and the molecular wire 240.

Example chemistries for linking the foundation oligos 224A, 224B to the contact terminals may include thiol chemistry (e.g., with electrodes containing gold), click chemistry, and/or metal-carbon (e.g., Au—C) covalent linkages. In some embodiments, the electrodes 210A, 210B can include gold deposited on a titanium adhesion layer on silicon dioxide (i.e., the “conventional” materials used to fabricate circuits). In some embodiments, the 3′ end of each of the single stranded oligos that make up the (double stranded) duplex can be functionalized (e.g., with a thiol group acting as a linker) to facilitate covalent bonding of the complex to the electrode 210A, 210B. By having the 3′ ends functionalized on both of the complementary oligos which make up the duplex, this means there is thiol functionality on both ends of duplex which facilitates the molecular bridge between the two electrodes to be made.

Click chemistry may include disposing a preliminary mixed monolayer of alcohol- and azide-terminated thiols on a gold surface, which may cause thiol to react with the gold to deliver the functional azide group to the gold surface. OCT-modified DNA can then be added and allowed to react with the azides to form a covalently tethered low-density DNA monolayer on gold. Metal-carbon covalent linkages may facilitate conjugation with p-orbital systems, thereby enhancing electrical conductivity through the linkage of the oligo.

Additionally or alternatively, the foundational oligo(s) 224A, 224B may be bonded to the electrode surface non-covalently via linkers 222A, 222B. In some embodiments, the linkers 222A, 222B may be a non-covalent linker. A non-covalent linker may be biological. In some embodiments, non-covalent linkages can be facilitated through protein-based binders. In some embodiments, the linkers 222A, 222B can include a protein complex. For example, one protein (e.g., biotin) may be covalently bound (via bond 221A, 221B) to an electrode 210A, 210B through a conventional chemistry (e.g., thiol-gold), and the foundation oligo 224A, 224B can be terminated (e.g., on a 3′ end) with a streptavidin such that the streptavidin-biotin links the oligo to the electrode 210A, 210B through a protein bridge. In some embodiments, pyrene, which similarly supports pi-pi stacking of orbitals between the linker 220A, 220B and the electrode 210A, 210B terminal may be used to couple the foundational oligo 224A, 224B to the electrodes 210A. 210B. This latter approach may support efficient electron transfer and therefore, forms a sufficiently conductive bridge from the bridge oligo 242 (e.g., from a biomolecule such as an enzyme bound to the bridge oligo 242) to the electrodes 210A, 210B. For example, in embodiments in which the electrodes may include carbon-based material (e.g., graphene), the DNA may be functionalized with pyrene to couple the DNA to the electrodes.

As shown in FIG. 2A, a portion of the bridge oligo 242 is single stranded (shown with dashed and dotted line). The portion of the bridge oligo 242 including the single stranded portion is therefore in a poor conductive state. The bridge oligo 242 may have a first binding region 244A configured to hybridize to at least a portion of the first foundation oligo 224A (e.g., an entire length of the first foundation oligo excluding the toehold region) and the second binding region 244B configured to hybridize to at least a portion of the second foundation oligo 224B (e.g., an entire length of the second foundation oligo excluding the toehold region).

For example, the first foundation oligo 224A may have a first sequence complementary to the first binding region 244A of the bridge oligo 240 and the second handle 224B may have a second sequence complementary to the second binding region 244B of the bridge oligo 240. Therefore, the first foundation oligo 224A and the first binding region 224A can hybridize to create a first double stranded portion of the molecular wire 240, and the second foundation oligo 224B and the second binding region 224B can hybridize to create a second double stranded portion of the molecular wire 240. In some embodiments, the bridge oligo 242 sequence may be configured such that after hybridization to the foundational oligos 224A, 224B, a portion of the first end of each of the foundational oligo(s) 224A, 224B (e.g., a portion adjacent to the linker molecules) includes single stranded DNA, as shown. In some embodiments, a number of bases in the foundation oligos 224A, 224B in a single stranded state may be predetermined. In some embodiments, no bases may be in a single stranded state (e.g., the first end portion and/or second end portion of the bridge oligo 242 may hybridize to a terminal end of the foundation oligos 224A, 224B). In some embodiments, a full length of the foundation oligo(s) 224A, 224B may be hybridized. In some embodiments, the more the base pairs in the foundation oligo(s) 224A, 224B that are hybridized, the higher the conductivity through the foundation oligo(s) 224A, 224B.

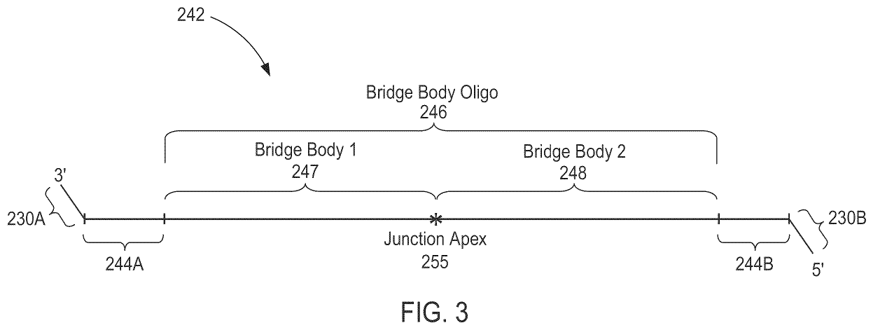

The bridge oligo 242 may include a first set of bases at a first end portion, which form a first toehold 230A and a second set of bases at a second end portion, which form a second toehold 230B. The structure of the bridge oligo 242 is described in further detail with respect to FIG. 3 below.

In some implementations, the device 200 can be structurally and/or functionally similar to the device 100, and therefore certain details of the device are not described herein again with respect to FIGS. 2A-2B.

FIG. 3 shows the bridge oligo 242 of a DNA wire including bridge body oligo (BBO) 246 that may be used in the device 100, 200 or any other device described herein. The bridge oligo 242 includes the first binding region 244A configured to hybridize to the first foundation oligo 224A (of FIG. 2A), and a second binding 244B region configured to hybridize to a second foundation oligo 224B (of FIG. 2A), according to an embodiment. The bridge oligo 242 may have a length sufficient to simultaneously bind to both foundation oligos 224A, 224A, each respectively bound to separate electrodes. In some embodiments, the bridge oligo 242 length may correspond to a length of each foundation oligo 224A, 224B, and a distance between each foundation oligo 224A, 224B. The spacing may be larger when the bridge oligo 242 is in the single stranded state than when the bridge oligo is in the double stranded state. For example, the bridge oligo 242 can have a reduced length when a duplex (dsDNA) is formed over the length of the 242 because double stranded (dsDNA) has about 0.34 nm spacing between bases in the dsDNA, and ssDNA has about 0.60 nm to about 0.7 nm spacing between nucleotides. Therefore, the length of the bridge oligo 242 may be sufficient to span the space defined between the foundation oligos 224A, 224B (e.g., and therefore span the space between the electrodes 210A, 210B) when the bridge oligo 242 is in double stranded form (i.e. is dsDNA). In some embodiments, a proportion of double stranded sections versus single stranded sections in the bridge oligo 242 may be configured such that the bridge oligo 3242 properly hybridizes to the foundation oligos 224A, 224B. In some embodiments, a ratio of the length of the bridge oligo 242 to a spacing or distance between the electrodes 210A and 210B is at least about 1:1.

In some embodiments, the bridge oligo 242 may be transformed into a duplex before the bridge oligo 242 is hybridized to the foundation oligos 224A, 224B. In some embodiments, the bridge oligo 242 may be transformed into a duplex after the bridge oligo 242 is hybridized to the foundation oligos 224A, 224B. SsDNA may coil or form secondary structures which may interfere with the ability to form duplexes. Therefore, in some embodiments, the bridge oligo 242 may be transformed to dsDNA before hybridizing to the foundation oligos because by converting the bridge oligo into dsDNA (forming a long duplex), the bridge oligo 242 may be prevented from coiling or forming secondary structures, and the bridge between the foundation oligos 224A, 224B may be formed more efficiently. In some embodiments, the bridge oligo 242 can have a sequence such that no secondary structures form. However, preventing secondary structures can become difficult as the molecular wire expands in size and integrates additional functionality (such as junctions to be described herein).

In some embodiments, and assembly of the molecular wire 242 can be performed in solution, which has advantages to employ thermal cycling and purification operations. The bridge oligo 242 can be configured to include portions including sequences that are complementary to a corresponding portion of the foundation oligo sequences. For example, the first binding region 244A of the bridge oligo 242 (excluding the toehold region) may have a sequence that is complementary to the first end of the first foundation oligo 224A, and the second binding region 244B of the bridge oligo 242 (excluding the toehold region) may have a sequence that is complementary to the first end of the second foundation oligo 224A. The complementary regions of the bridge oligo 242 may be proximal or close to the terminal ends of the bridge oligo 242. In some embodiments, the bridge body oligo 242 can include a bridge body oligo (BBO) 246, which may not contain complementary sequence(s) to the foundation oligos 224A, 224B. In other words, a portion of the bridge oligo 242 in between the binding region 244A to the first foundation oligo 224A and the binding region 244B to second foundation oligo 224B may not include complementary sequences to the first foundation oligo 224A and the second foundation oligo 224B, respectively, to prevent undesirable hybridization. The bridge oligo 242 may include toehold regions 230A and 230B on both terminal ends of that can support toehold mediated strand displacement actions. The bridge body oligo 246 may include a first bridge body 247 and a second bridge body 248 that define a junction apex 235 therebetween, which can be used for integrating functional probes in certain embodiments, as described in further detail below.

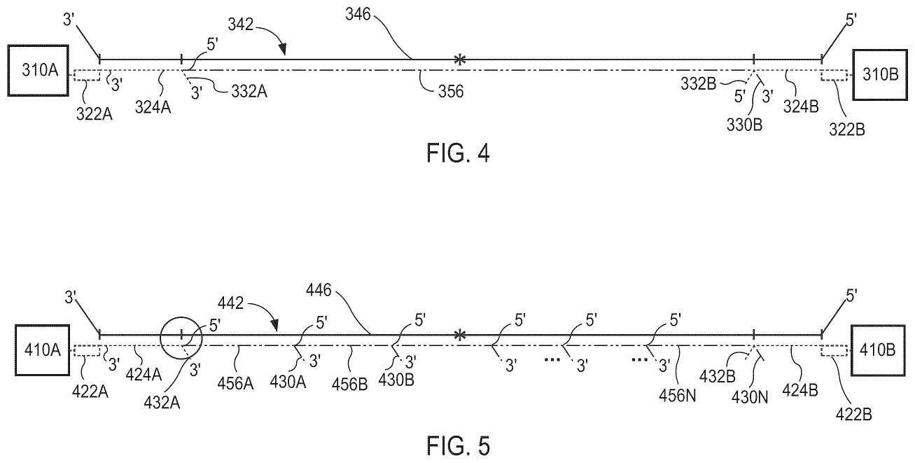

In some embodiments, the circuit between the first electrode and the second electrode may be completed by integrating the complement(s) (e.g., the complementary strand(s) of DNA) to the bridge body oligo 246). In other words, a strand of DNA having a sequence complementary to a portion of the bridge oligo 242 extending between the first binding region 244A and the second binding region 244B (e.g., the bridge body complement 246) can be hybridized to the portion of the bridge oligo 242, thereby forming a double stranded bridge between the two contact terminals, as shown in FIGS. 4-5.

FIG. 4 shows a molecular wire including a bridge body oligo 346 including a bridge body complement (BBC) 346 extending between two linkers 322A, 322B. FIG. 5 shows a molecular wire including a collection of serial complements 456A, 456B, 456C, . . . 456N to the bridge body oligo 446. As shown in FIG. 4, the device may include a fourth oligo to facilitate completion of the molecular wire referred to as the bridge body complement (BBC) oligo 352. The bridge body complement 352 may have a sequence that is complementary to the bridge body portion 346 of the bridge oligo 342 such that at least a portion of the bridge oligo 342 is entirely double stranded. In some embodiments, when the bridge body complement 356 is hybridized to the bridge body oligo 346, all bases are bound to their complements, forming a continuous double stranded DNA linkage between the two terminals 310A, 310B. Conversely, if there are missing bases in the resulting bridge, the conductivity of the molecular wire may be reduced. The presence, or lack thereof, of nicks in the dsDNA may not compromise the electrical properties of the molecular wire. In some embodiments, the bridge oligo 342 may be converted to its double stranded state prior to binding to the foundation oligos 324A, 324B.

As shown, the linkers 322A, 322B can include a foundation oligo 324A, 324B extending therefrom and including toe hold regions 332A, 332B. In some embodiments, the bridge body complement oligo 356 can hybridize to the bridge body oligo 346 such that a first single stranded binding region of the bridge oligo 342 extends beyond a first end of the bridge body complement oligo 356 and a second single stranded binding region of the bridge oligo 342 extends beyond a second end of the bridge body complement oligo 356. The first binding region can be configured to hybridize to the first foundation oligo 324A, and the second binding region can be configured to hybridize to the second foundation oligo 324B such that the DNA of the molecular wire 342 between the first linker 320A and the second linker 320B is double stranded.

In some embodiments, the completed molecular wire may include toeholds 330B present on one or both ends of the bridge body oligo 346 and toeholds 332A, 332B present on the terminal ends of each foundation oligos 324A, 324B. As shown in FIG. 5, the bridge body complement can include or be formed from a collection of shorter oligos 456A, 456B, . . . , 456N spanning between the linkers 422A, 422B, each bridge body complement oligo having a toehold 430A, 430B, . . . 430N. In some embodiments, the bridge body complement 456A-456N may include toeholds on one or both ends thereof. In some embodiments, all bases along the bridge oligo 442 may have their respective complement hybridized thereto. Therefore, through hybridizing the bridge body complements 456A-456N to the bridge body 446, the two electrode terminals 410A, 410B are connected via a dsDNA bridge. When all bases are correctly paired, the two electrode terminals 410A, 410B can facilitate a current flow therethrough when a potential is applied to the therebetween.

In some embodiments, the bridge oligo 342, 442 may include one or more functional groups or functional moieties to modify conductivity through the molecular wire, as described herein. In some embodiments, the bridge may include a functional moiety (e.g., similar to transduction agent 180) on every nucleotide. In some embodiments, the functional moieties may be integrated at a predetermined spacing. For example, the functional moieties may be integrated on every other nucleotide in the wire. In some embodiments, a spacing between adjacent functional moieties may be every 2, every 3, every 4, every 5, every 6, every 7, every 8, every 9, every 10, every 20, every 30 nucleotides. In some embodiments, the spacing between adjacent functional moieties may be on every nucleotide to every 4 nucleotides, inclusive of all ranges and subranges therebetween (e.g., spacing about equal to length of one nucleotide, two nucleotides, three nucleotides, or 4 nucleotides, inclusive). In some embodiments, one or more properties of the molecular wire may correspond to a frequency of the functional moieties and/or a type of functional moieties. The functional moieties may include any of the functional moieties described herein (e.g., aromatic rings, metal atoms, etc.) The functional moieties may be integrated into the nucleotide using any of the methods described herein (e.g., substitution).

FIG. 6 shows a device 500 including a DNA wire including a genotyping sensor region (GSR) 560, according to an embodiment. As shown, the molecular wire 540 includes the bridge body oligo 542 and a plurality of bridge body complement oligos 556A, 556B, . . . 556N, 556(N+1) hybridized thereto. A first binding portion of the bridge body oligo 542 can hybridize to a first foundation oligo 524A, and a second binding portion of the bridge body oligo 542 can hybridize to a second foundation oligo 524B. The first foundation oligo 524A can have first toe hold region 532A, and the second foundation oligo 524B can have second toe hold region 532B. The first foundation oligo 524A and the second foundation oligo 524B can be coupled to the first electrode 510A and the second electrode 510B, respectively, via first linker 522A and second linker 522B. To integrate the sensing element 560, a gap may be inserted in the dsDNA molecular bridge to serve as a probe for genotyping or gene expression. In some embodiments, the sensor may be formed based on the dsDNA molecular wire 540 by including a region of the bridge oligo 542 which is maintained in the single stranded state and can serve as a genetic probe. The single stranded region can be designed to be of any suitable length of nucleotides. For example, the single stranded region may include between about 3 nucleotides to as many as about 30, 50, 100 or more nucleotides. In some embodiments, the single strand region may be configured such that the genomic information can be probed (e.g., the ssDNA region may include appropriate/representative fragment lengths). In some embodiments, the optimum length of the ssDNA region may be determined based on a sample being probed. In some embodiments, a sequence of the single stranded region may be complementary or correspond to a binding region of a sample to be probed. Therefore, the GSR 580 may function to measure a known sequence in a biological sample (e.g., may function as a genotyping (SNP) probe).

By creating a zone in the bridge oligo 542 which is single stranded, the ability of the molecular wire 540 to efficiently conduct electricity between the two electrodes has been disrupted or reduced. The conductivity of the molecular wire 540 may increase upon binding of a target sequence or molecule, resulting in a detectable signal between the electrodes 510A, 510B. For example, a voltage can be applied to the two electrodes 510A, 510B and a current (e.g., a baseline or background current) moving through the molecular wire 540 can be measured when the single stranded zone is present in the wire. Upon exposure of this sensor to a biological sample, e.g., if a complement to the GSR 680 is present in the biological sample, hybridization occurs, resulting in the creation of a continuous double stranded bridge between the two electrodes 510A, 510B. As a result of completion of the dsDNA bridge, the current flowing between the electrodes 510A, 510B increases, indicating that the complement is present in the sample. The increase in observed current can allow for the genotyping call.

In some embodiments, a device may include a plurality of sensors, and multiple copies of each probe may be disposed on each electrode, increasing the signal to noise ratio in making a genotyping call. Additionally or alternatively, a device may include multiple sensors on independent electrode pairs, thereby enabling (i) the quantification of the presence, or lack thereof, of a particular sequence and/or (ii) the ability to simultaneously count (e.g., digitally) the number of electrode pairs measuring the target sequence to determine the level of expression of that particular sequence. In some embodiments, the device may include a plurality of single stranded regions on a single bridge oligo, each having a unique genetically relevant sequence and then a measurement of an increased current can indicate that both (or more than two) of the probe sequences are present in a sample simultaneously.

In some embodiments, the device may include an electrode pair that includes a cluster of molecular wires, each having the same probe sequence. Alternatively, the device may include an array of electrode pairs, each having clusters of unique probe sequences. Alternatively, the device may include an array of electrode pairs, with each electrode pair having a single bridge oligo and unique GSR, resulting in an array of single molecule sensors.

In some embodiments, the molecular wire may be configured to read an unknown genetic sequence. To enable the molecular wire to read an unknown DNA sequence, a DNA junction may be inserted into the nanostructure. A DNA junction may be defined as a location where 3 or more double helices of DNA come together. The purpose of the junction can be to facilitate integration or coupling of protein-based probes (e.g., a polymerase, aptamers, etc.). In some embodiments, a polymerase may be used for DNA sequencing applications. In some embodiments, other probes may be used such as, single stranded DNA configured as a genotyping probe for unknown sequences, an aptamer for protein or small molecule detection, an antibody for protein detection, etc. In some embodiments, the junction may be configured to host a pair of binders, enabling a sandwich-based measurement of a target molecule (e.g., sandwich ELISA).

In some embodiments, a stable junction (e.g., chemically and/or physically stable) can be formed when three (or more) sets of (double stranded) oligonucleotides are brought together. The sequences can be designed in such a way that upon assembly and/or through hybridization, a three-way intersection of double stranded DNA branches is created, as shown in FIG. 7. In some embodiments, the junction may be configured such that it does not migrate (move). In other words, the junction may be migrationally immobile (e.g., reducing and/or eliminating the possibility of branch point migration). In some embodiments, the junction may include a sequence having unique trimer units and/or unique tetramer units to prevent migration of the junction along a length of the molecular wire and/or migration off of the molecular wire.

In some embodiments, the junction may include more than three sets of oligonucleotides. Additional oligos may simplify or enhance the performance the coupling of the junction-based nanostructure to the electrodes and simplify the integration of probes (e.g., a polymerase). Including additional oligos in the junction may also enable regeneration of the sensor(s) using toehold mediated strand displacement. Although FIGS. 4-6 show toeholds on the 3′ end of the BBC oligos, in some embodiments, the BBC oligos may include toeholds on both the 3′ end and the 5′ end.

FIG. 7 shows a device 600 including junction-based nanostructure. The device 600 may include a first foundation oligo 624A and a second foundation oligo 624B linked (e.g., via linker molecules 622A, 622B, respectively) to a first electrode 610A and a second electrode 610A. The device 600 may further include a bridge oligo serving as a backbone connecting the two electrodes 610A, 610B. The bridge oligo may include a first bridge body 647 and a second bridge body 648 that meet at a point, referred to herein as the junction apex 635. The bridge oligo may be coupled to one or more bridge body complement oligos 656A, 656B, 656C, 656D to form a dsDNA. In some embodiments, the bridge body complement oligos 656A-656D can be modified such that each bridge body complement includes an extension that is complementary to the other bridge body complement extension, but not complementary to the bridge oligo. A first set of bridge body complement oligos 656A, 656B may form a first bridge junction arm (BJA1) 657 and a second set of bridge body complement oligos 656C, 656D may form a second bridge junction arm (BJA2) 658. Each of the bridge junction arms 657, 658 may meet at the junction apex 655. The junction apex 655 may be located approximately at the midpoint of the bridge oligo, and the extensions of each bridge junction arm 657, 658 that are not complementary to the bridge oligo may diverge from the bridge oligo at the junction apex 655.

As shown in FIG. 7, the hybridization of the bridge oligo with a portion of the bridge oligo complement, and hybridization of the bridge junction arms 657, 658 forms a double stranded DNA complex where all bases have hybridized to their corresponding complementary bases. In some embodiments, a plurality of oligos may be hybridized to the bridge oligo to ensure all bases of the bridge oligo are hybridized to a complement. For example, the bridge junction arms 657, 658 may include a plurality of oligos. In some embodiments, each of the plurality of oligos may include a toehold region. The extensions of the bridge junction arms 657, 658 are hybridized to each other, thus resulting in a double stranded arm extending away from the bridge oligo (e.g., at the junction 635). The bridge junction arms 657, 658 may each have toehold regions on a first end closest to the bridge oligo (proximal to the complementary region of the foundation oligo), and may not have toeholds on a second end that forms the non-terminated end of the double stranded arm extending from the junction 635. The bridge junction arms 657, 658 may each have oligo extensions which allow for hybridization of functional probes 680 onto a portion of the molecular wire (e.g., the double stranded arm extending from the bridge oligo) that is not electrically connected to the electrodes 610A, 610B.

In some embodiments, the branched zones around the junction may not include long stretches (e.g., long stretches including about 5 bases to about 20 bases, inclusive) of guanine bases. In other words, the junction zones may not include G2 repeats due to the thermodynamic properties of guanine. In some embodiments, the extensions or double stranded arm portion may have a minimum of about 6 nucleotides. In some embodiments, a larger number of nucleotides or base pairs in the extensions may increase thermal stability (e.g., stability at high temperatures). In some embodiments, the oligonucleotide strands may be designed based on bonding properties of the nucleotides. GC pairs have stronger bonds than AT pairs (e.g., due to a larger number of bonds in GC pairs). In some embodiments, the oligonucleotide strands may have sequences such that GC pairs are at predetermined locations (e.g., junction zones may not include a large number of G's, terminal ends of strands may include GC pairs, etc.) In some embodiments, the extensions (e.g. the portion of the bridge junction arm that extends away from the bridge oligo) may alternate between purines (e.g., A's and G's) and pyrimidines (e.g., T's and C's). In other words, the extensions may not include or may have limited purines adjacent to one another (e.g., AGAGAG) and/or pyrimidines adjacent to one another (e.g., TCTCTC). In some embodiments, a terminal end of the sequence may include GC pairs to avoid fraying.

FIG. 8 shows a close-up view of the device 600 of FIG. 7 including a functional probe (or transduction agent) hybridized to the double stranded arm extending from the junction 635. In some embodiments, the bridge junction arms 657, 658 include a region (e.g., about 0 to 20 bases or 0 to 5 bases) that may be non-complementary to each other, thereby supporting a small spatial separation between the terminal ends of each extension of the arm, as shown in FIG. 8. Each of the bridge junction arm terminal ends may be bound to specific points on and/or within the functional probe 680 (e.g., a polymerase). For example, the unpaired region of the bridge junction arm terminal ends serve as linkage regions for integrating a probe 660. It is desirable to reduce or minimize the length of the unpaired extensions on each bridge junction arm. In some embodiments, the length of the two extensions are about the same length for the two arms. In some embodiments, the lengths of the unpaired extensions are different for the two arms.

Through the integration of the functional probe 660, current can be modulated as the functional probe 680 (e.g., the polymerase) undergoes conformational changes. For example, the molecular wire can sense changes in current in response to the polymerase incorporating bases into a ssDNA molecule and converting the ssDNA into dsDNA. More specifically, as a voltage is applied to the pair of electrodes (not shown), current can flow through the DNA nanostructure, with a first portion of the current flowing direct across the junction 655 between the two electrodes and a second portion of the current flowing through the first extension (e.g., a portion of bride junction arm 657), the polymerase, and the second extension (e.g., a portion of bridge junction arm 658),, which collectively form a parallel branch of the circuit. In other words, the second portion of current may flow up the first extension to the polymerase, and then flow back down the second extension to the second electrode. As the polymerase performs its natural operation of incorporating nucleotides onto a single stranded oligo and converting the oligo into double stranded DNA, the conformation changes induce corresponding changes in the current flowing through the polymerase, which can be measured as changes in a total current flowing between the electrodes. In some embodiments, the nanostructure may be configured to direct a predetermined portion of current to the polymerase, as described in detail below.

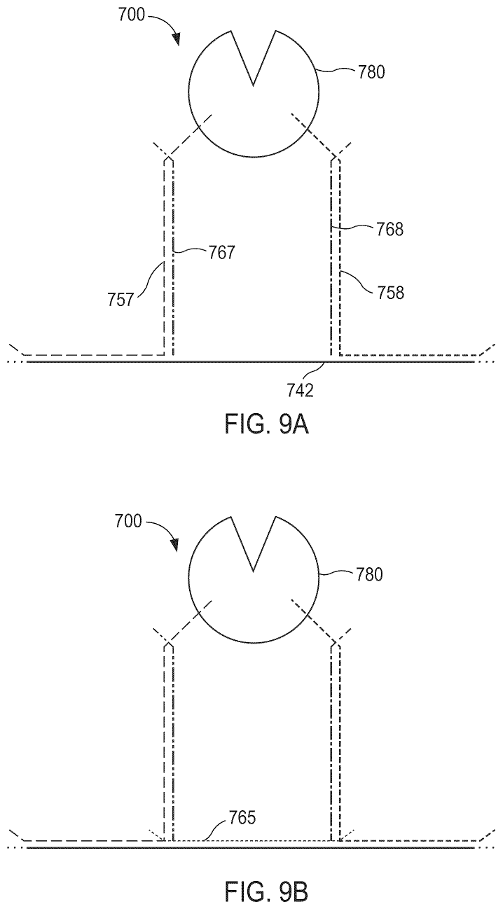

FIGS. 9A-9B show a device 700 including a sensor probe 760 coupled to the bridge oligo via bridge junction arms 757, 758 that are not complementary to one another, according to an embodiment. As shown, the molecular circuit of device 700 includes two junctions arms, each of which are orthogonal to the other and/or spaced a predetermined distance such that the junctions cannot bind to one another

The device 700 includes a nanostructure 765 at the junction that enables the path of least resistance to be through the functional probe 780 (e.g., polymerase) and not directly over the junction. Therefore, a portion of current is directed through the functional probe 780. In some embodiments, the portion of current directed through the functional probe 780 may be larger than a portion of current directed over the junction (e.g., directly between the electrodes).

As shown, the bridge body complement oligos from bridge junction arms 757, 758 may be configured such that when hybridized to the bridge oligo 742, a gap exists between the branch off points of the junction arms 757, 758, thereby creating two junction apices. For example, the extensions of the bridge junction arms 757, 758 which are not complementary to the bridge oligo may also not be complementary to each other. To form double stranded arms, oligos 767, 768 that are complementary to each of the extension portions of the bridge junction arms 757, 758 can be hybridized to the extension portions of the bridge junction arm 757, 758, to form a continuous double stranded DNA from the electrodes up to the functional probe 780 for both bridge junction arms 757, 758. This hybridization allows minimization (e.g., elimination) of the extension of the bridge junction arm 757, 758 that is single stranded, as conjugation of the bridge junction arm 757, 758 to the functional probe 780 may not be hindered through the bridge junction arm 757, 748 being double stranded in nature. In some embodiments, each of the complementary junction arms 757, 758 may include one or more toeholds (e.g., at a first end and/or a second end thereof).

Each of the bridge junction arms and/or the complements to the bridge junction arms are conjugated to specific points within the functional probe (e.g., the polymerase), inducing the functional probe to be an integral component of the circuit. The functional probe may be any suitable functional probe such as a polymerase including, for example, Phi 29, and/or mutants of Phi 29. In some embodiments, only the bridge junction arms are conjugated to the functional probe. In some embodiments, only the complements to the bridge junction arms are conjugated to the functional probe. In some cases, it may be advantageous to have the complements to the bridge junction arms conjugated to the functional probe. For example, it may be advantageous such that the polymerase can be more easily or efficiently swapped in and/or out (e.g., replaced) with other probes that have the same complements conjugated thereto. The removal of a probe can be done by strand displacement (e.g., if the bridge junction arm complements have toehold regions), and the new probe can be integrated into the circuit upon conjugation of one or more of the bridge junction arm complements which include the appropriate sequence to a portion of the new probe.

In some embodiments, if the complement to the bridge junction arms are conjugated to the functional probe 780, the bridge junction arms 757, 758 may each have toeholds on both ends (e.g., the terminal end closer to the functional probe and the terminal end closer to the respective electrode). Conversely, if the bridge junction arm 757, 748 is conjugated to the functional probe 780, the complements to the bridge junction arms may include toeholds (e.g., on both ends). In FIG. 9A, a segment of the bridge oligo extending between the two apices is single stranded, thus reducing and/or preventing current flow across this segment of the bridge oligo. As shown in FIG. 9B, the segment of the bridge oligo between the two apices may be hybridized to an apex bridge 765. The apex bridge 765 can be configured to regulate the current going into the branch of the circuit hosting the polymerase. The apex bridge 765 can serve as a quality control check on each molecular circuit. For example, the apex bridge 765 may be configured to cause a majority or a totality of the current to flow therethrough (e.g., as if the polymerase 780 was not integrated into the circuit) such that current can be measured and operation of the circuit can be confirmed. The length of the apex bridge region 765 can be reduced to as few as 1 base, or extended to be up to as long as the length or the bridge oligo 742. In some embodiments, the apex bridge 765 can include a sequence and/or functional groups coupled thereto such that the apex bridge 765 has a resistance greater than a resistance across the electrical path through the functional probe 780.

In some embodiments, the molecular sensor(s) may be configured to be regenerated to maximize the lifetime of the microfabricated sensor array, as manufacturing and disposing of a device after a single use makes the measurement process (prohibitively) expensive. In some embodiments, the molecular sensor may have a functional surface (i.e., a surface chemistry) which enables the ability to be reset (e.g., the components that have been engineered at the molecular level can be removed and re-grown from a substrate, such as the electrode array). For example, the surface chemistry may enable a process of being “erased” or removed and then deposited and/or grown again, either in full or partially. In some embodiments, one or more of the oligos in the molecular wire may include toeholds to facilitate sensor regeneration via toehold mediated strand displacement.

FIG. 10 shows an example of a toehold mediation strand displacement chemical reaction. In this figure, N* represents the complementary strand of N. Initially, a first oligonucleotide strand S1 may include a first portion (including sequence 2*) coupled to a portion (including sequence 2) of a second oligonucleotide strand S2. The first oligonucleotide strand S1 may further include a toehold portion (including sequence 3*). A third oligonucleotide S3 may include a first portion (including sequence 2) and a toehold portion (including sequence 3). During branch migration, the toehold portion (sequence 3) of the third oligonucleotide strand S3 may hybridize to the toehold portion (sequence 3*) of the first oligonucleotide strand S1 and ultimately begin to replace the second oligonucleotide strand S2, as shown. During strand dissociation, the first portion (sequence 2) of the third oligonucleotide strand S3 may be hybridized to the first portion (sequence 2*) of the first oligonucleotide strand S1, and the toehold potion of the third oligonucleotide strand may be hybridized to the toehold portion of the first oligonucleotide strand S1 such that a duplex is formed and the second oligonucleotide strand S2 is fully displaced.

In some embodiments, the molecular wires described herein may include toeholds on each of the oligonucleotides such that the oligonucleotide strands may be replaced via toehold mediated strand displacement. Toehold mediated strand displacement may allow regeneration such that no fouling of the underlying materials occur or may allow regeneration many times (e.g., potentially infinite number of times) before the underlying electronics don't support redeposition (e.g., due to surface degradation, corrosion, etc.). In some embodiments, the oligonucleotides in the molecular wire may be configured such that upon erasing/removal of the functional surface, there is no residual chemistry or measurable signal that is reminiscent of the functional surface that was previously there. In some embodiments, the oligonucleotides in the molecular wire may be configured such that upon regeneration, a difference in performance between one functional surface and the new functional surface is minimized. In other words, the device performance, after the functional surface is formed, meets performance specifications independent of it being the first time the surface is created or the Nth time, where N is greater than 1. For example, toehold strand displacement may be repeated several times (infinite number of reuses) without any indication that the device has a history of use.

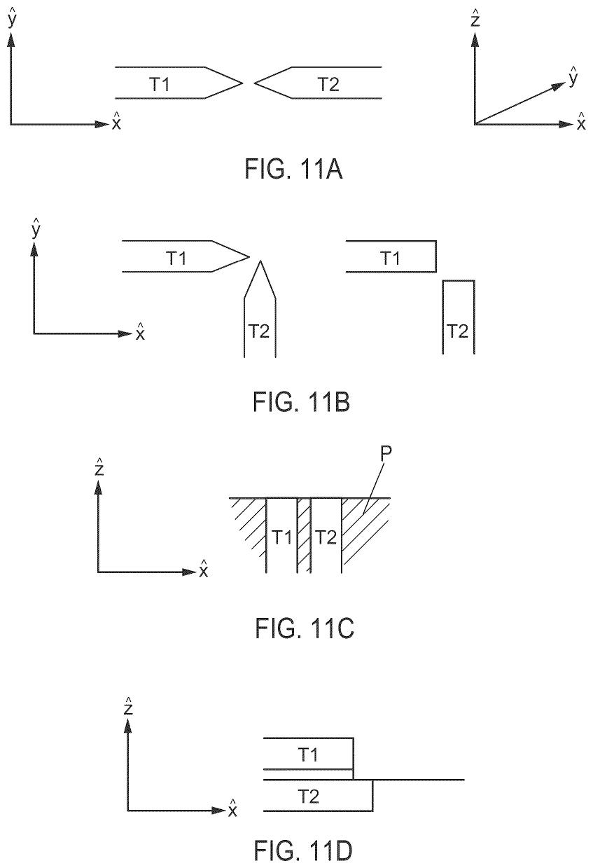

FIGS. 11A-11D show different open circuit configurations of a first electrode terminal and a second electrode terminal, according to embodiments. As shown, the first electrode terminal T1 and the second electrode terminal T2 may be coplanar to one another. In some embodiments, the electrode terminals T1, T2 may taper or include an apex, as shown. In some embodiments, the first electrode terminal T1 and the second electrode terminal T2 may be orthogonal to one another. The electrode terminals T1, T2 may have a tapered end and/or may include a flat terminal end. As shown in FIG. 11C, the first electrode terminal T1 and the second electrode terminal T2 may be stacked in a first configuration in which the electrode terminals T1 are adjacent to one another along an x axis (e.g., stacked in the z-direction as shown in FIG. 11C). In some embodiments, the body of the electrodes are buried under the passivation materials (labeled “P”), thus allowing for connection between the two terminals to be on the planar surface labeled as the x axis. As shown in FIG. 11D, the first electrode terminal T1 and the second electrode terminal T2 may be stacked in a second configuration in which the electrode terminals T1 are adjacent to one another along a z axis. The two terminals may be linearly stacked (not illustrated) or offset relative to one another. In some embodiments, the electrode terminals may have a predetermined distance therebetween. In some embodiments, the distance between the electrode terminals T1, T2 may be in a range of about 20 nm to about 200 nm, inclusive of all ranges and subranges therebetween.

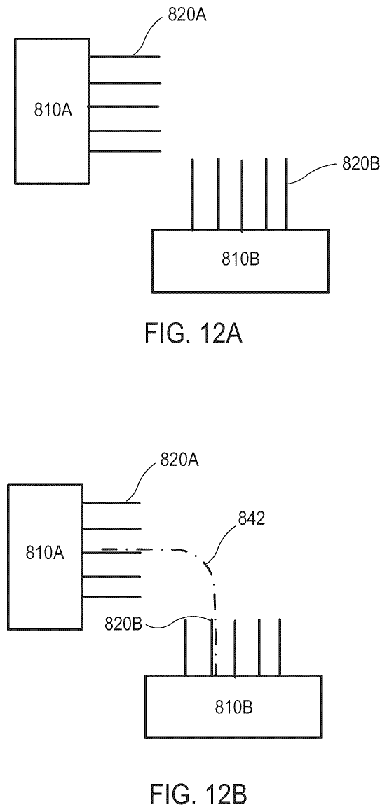

FIG. 12A shows electrode terminals 810A, 810B with linker strands 820A. 820B extending therefrom, and FIG. 12B shows a bridge oligo 842 connecting the linker strands 820A. 820B, according to embodiments. As shown in FIG. 12A, a first electrode terminal 810A and a second electrode terminal 810B may be arranged orthogonal to one another and each include a plurality of linker molecules 820A, 820B. In some embodiments, a bridge oligo 842 may be configured to hybridize to the linker molecules 820A, 820B to electrically connect the first and second electrodes 810A. 810B. In some embodiments, the electrode terminals 810A, 810B may include the same covalent linkages. In some embodiments, the electrode terminals 810A, 810B may include covalent linkages that are different from each other. In some embodiments, the electrode terminals 810A, 810B can have foundation oligos extending therefrom that can either have the same or different sequences. In some embodiments, the foundation oligos extending from the same electrode may include the same sequence.

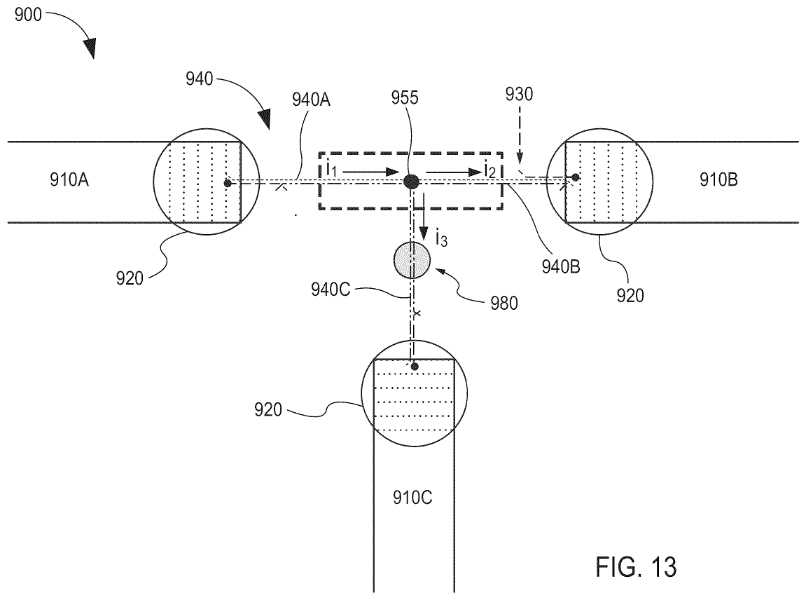

FIG. 13 shows a schematic of a molecular circuit including three terminal electrodes 910A, 910B, 910C, with a molecular wire 940 (e.g., double stranded DNA-based molecular wire). The molecular circuit includes a first electrode 910A (e.g., source), a second electrode 910B (e.g., drain), and a third electrode 910C (e.g., drain). The electrodes 910A-910C can be electrically connected by the molecular wire 940 that can include a junction 955 and three branches 940A, 940B, 940C. In some embodiments, the molecular wire 940 can include dsDNA and/or DNA origami. In some embodiments, the molecular wire 940 can be coupled to the electrodes 910A-910C via one or more linkers 920 coupled to the electrodes 910A-910C. For example, each electrode 910A-910C can include one or more linkers 920 disposed on a surface thereof. The position of each branch of the molecular wire 940 is determined through the use of DNA origami, which can also serve as the conductive bridge between the top-down fabricated electrode and the bottom-up self-assembled molecular circuit. For example, each electrode terminal 910A-910C can include a DNA origami coupled thereto, the DNA origami including a plurality of programmable attachment sites configured to hybridize to a predetermined sequence of DNA. The molecular wire 940 can include single stranded binding regions including the predetermined sequence of DNA and configured to hybridize to a programmable attachment site of the DNA origami. In some embodiments, a portion of a first branch 940A of the molecular wire 940 can be connected to the first electrode 910A, a portion of a second branch 940B can be connected to the second electrode 910B, and a portion of a third branch 940C can be connected to the third electrode 910C.

Each of the dsDNA circuit elements (e.g., each branch 940A-940C) have toehold strand extensions 930, which can be used to enable programming (and reprogramming) of the molecular circuits via toehold mediated strand displacement. Programming of a circuit is accomplished through the incorporation and/or replacement of branches of the molecular circuit with different catalytic operators. The position of each operator is determined by the design sequence of the molecular wire 940. In addition, through the use of toehold mediated strand displacement, each molecular circuit can be removed and replaced with new, user-defined molecular circuit elements without adversely impacting the underlying substrate components. This allows for the underlying CMOS architecture to remain fixed while the molecular circuitry is configurable for the intended application.

In some embodiments, the molecular wire 940 can include a catalytic operator 980 (i.e., a transduction agent) such as a polymerase coupled to a first branch of the molecular wire 940. In some embodiments, the catalytic operator 980 is in a first state or conformation when a target molecule is not bound to the catalytic operator 980. When the catalytic operator 980 is in the first state, the third branch 940C can have a lower conductivity than the second branch 940B, and therefore, a current is through the third branch 940C is lower than a current i₂ through the second branch 940B. When the target molecule binds to the catalytic operator 980, the catalytic operator 980 can undergo a conformation change such the third branch 940C increases in conductivity, and a current is through the third branch 940C of the molecular wire 940 increases. In some embodiments, the second electrode 910B can act as a reference electrode (e.g., for troubleshooting and/or normalization of signal). The molecular circuit 900 can be structurally and/or functionally similar to the devices 100, and therefore, certain details of the molecular circuit 900 are not described herein with respect to FIG. 13.

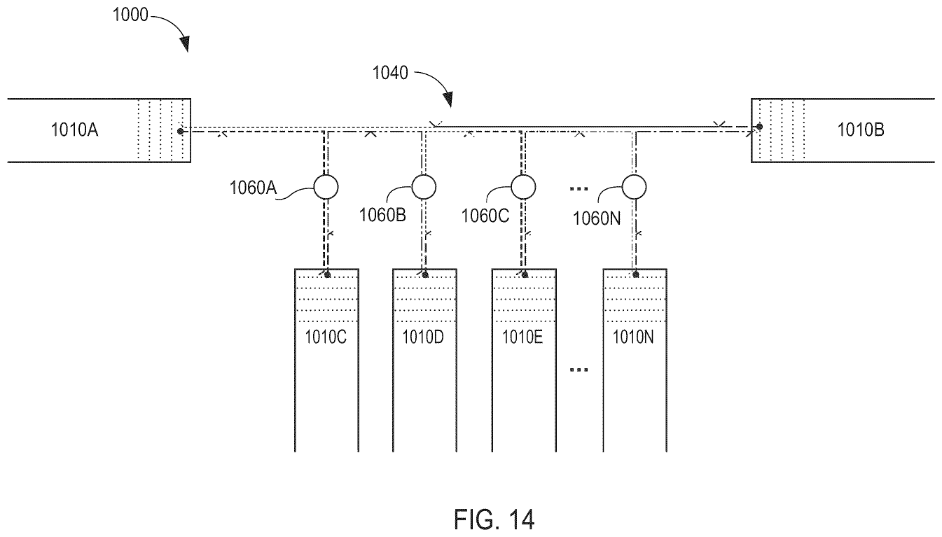

FIG. 14 is a schematic of the device of FIG. 13 scaled to include a plurality of catalytic operators (or transduction agents) 1060A, 1060B, 1060C, . . . , 1060N. As shown, the device 1000 includes a unit cell that includes N electrode terminals 1010A, 1010B, 1010C, 1010D, 1010E, . . . , 1010N, where N is greater than three, and each catalytic operator 1060A-N is organized in such a way that a collective suite of operations, such as a multi-step chemical synthesis process, can be performed. Specifically, the catalytic operators can include proteins, enzymes or the like. In some embodiments, each catalytic operator 1010A-1010N may be configured to utilize a product or biproduct of a reaction of the previous catalytic operator. For example, catalytic operator 1060A may perform a chemical process resulting in first product, and catalytic operator 1060B may be configured to perform a chemical process using the first product (e.g., as a reactant or a catalyst). In some embodiments, the chemical process can be monitored based on a current directed through each catalytic operator, and therefore, an electrical signal measured between a subset of the electrodes. For example, a change in current measured between a given pair of electrodes can be measured indicating that a particular step of a chemical process has occurred. As with the three-terminal device, the junction architecture and ordering of the operators can be controlled through the definition of the nucleic acid sequences that are conjugated to each operator 1060A-1060N. Additionally, each molecular wire 1040 may be localized within the device based on a sequences of the molecular wire 1040 that correspond to sequence of one or more programmable sites of a DNA origami disposed on particular electrode 1010A-1010N. The molecular circuit 100 can be structurally and/or functionally similar to the devices 100, 900, and therefore, certain details of the molecular circuit 100 are not described herein with respect to FIG. 14.

FIG. 15 is a schematic block diagram of an example method 1100 of building a molecular circuit (e.g., any of the devices 100, 200, 500, 600, 700, 900, 1000 described herein), according to an embodiment. In some embodiments, the method 1100 includes coupling a first linker molecule to a first electrode and a second linker molecule to a second electrode, the first linker molecule including a first staple (or site) and the second linker molecule having a second staple (or site), at 1110. In some embodiments, the first and second electrodes can be disposed on a semiconductor chip. In some embodiments, the first and second electrodes can be part of an electrode array including a plurality of electrodes. In some embodiments, the first staple and the second staple can include programmable sites. For example, the first staple and the second staple can include DNA molecules including a predetermined sequence of DNA. In some embodiments, at least one of the first linker molecule and the second linker molecular can include a DNA molecule and/or DNA origami. In some embodiments, at least a portion of the DNA molecule and/or DNA origami can be double stranded.

The method 1100 can include incorporating a plurality of functional groups into a DNA molecule of a molecular wire at predetermined positions along the DNA molecule, at 1112. In some embodiments, incorporating the plurality of functional groups to the DNA molecule can include substituting one or more nucleotides of the DNA molecule with modified nucleotides including the functional groups attached thereto. In some embodiments, the functional groups can improve conductivity of the DNA wire. In some embodiments, the functional groups can include any of the functional groups described herein (e.g., functional groups 170).

In some embodiments, the method 1100 can optionally include incorporating the functional groups into at least one of the first linker molecule or the second linker molecule to increase conductivity of the interface between the first and second electrodes and the first and second linker molecules. In some embodiments, the method 1100 includes, hybridizing a first binding region of the molecular wire to the first staple of the first linker and the second binding region of the molecular wire to the second staple of the second linker, at 1114. In some embodiments, the molecular wire can include a portion that is dsDNA, and the first binding region and the second binding region can include ssDNA. In some embodiments, the first binding region and the second binding region can be configured to include sequences complementary to the sequences of the first staple of the first linker and the second staple of the second linker, respectively.

In some embodiments, the method 1100 can include, hybridizing a third binding region of the molecular wire to a transduction agent (e.g., a catalytic operator such as a polymerase), at 1116. In some embodiments, the transduction agent can include any of the transduction agents described herein (e.g., transduction agent 180). In some embodiment, the method 1100 can include sensing, for example, when the transduction agent undergoes a chemical event, a change in current between the first electrode and the second electrode, at 1118. In this way, chemical events involved in processes such as DNA synthesis and/or binding to target molecules in an environment can be monitored. In some embodiments, the method 1100 can include determining one or more characteristics of the chemical event based on the change in current, at 1120. For example, the method can include determining presence or concentration of a target molecule (e.g., protein), determining a sequence of a target DNA molecule, determining a sequence of a protein molecule, etc. in the environment based on the change in current between the electrodes.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus, comprising:
a first electrode and a second electrode defining a gap therebetween;
a molecular wire disposed across the gap, and configured to electrically connect the first electrode and the second electrode, the molecular wire including:
at least one molecule of deoxyribonucleic acid (DNA) including a double stranded portion; and
a plurality of functional groups coupled to nucleotides of the at least one molecule of DNA at predetermined positions along the at least one molecule of DNA, the plurality of functional groups configured to cause electron delocalization along the molecular wire to increase an electrical conductivity of the molecular wire; and
a first molecular linker configured to couple a first portion of the molecular wire to the first electrode and a second molecular linker configured to couple a second portion of the molecular wire to the second electrode.

2. The apparatus of claim 1, wherein the plurality of functional groups include aromatic rings, the aromatic rings spaced along the molecular wire such that a pi orbital of each aromatic ring overlaps with a pi orbital of an adjacent aromatic ring.

3. The apparatus of claim 1, wherein the plurality of functional groups include a metal atom.

4. The apparatus of claim 1, wherein the at least one molecule of DNA of the molecular wire includes a DNA origami structure.

5. The apparatus of claim 1, wherein at least one of the first molecular linker and the second molecular linker include a DNA origami linker, the DNA origami linker including a plurality of sites each having a predetermined DNA sequence.

6. The apparatus of claim 5, wherein the molecular wire includes a first binding region configured to hybridize to a first site of the plurality of sites, and a second binding region configured to hybridize to a second site of the plurality of sites to couple the molecular wire to the first electrode and the second electrode.

7. The apparatus of claim 1, further comprising:
a transduction agent coupled to the molecular wire, the transduction agent configured to change a current directed between the first electrode and the second electrode in response to a chemical event occurring in an environment surrounding the transduction agent.

8. The apparatus of claim 7, wherein the transduction agent includes a polymerase, the polymerase configured to undergo a conformation change in response to reacting with a nucleotide in the environment binding to a binding site of the polymerase, the conformation change of the polymerase configured to cause the change in current directed between the first electrode and the second electrode.

9. The apparatus of claim 1, wherein at least one of the molecular wire, the first molecular linker, or the second molecular linker include toehold regions such that one or more portions of the molecular wire can be replace via toehold mediated strand displacement.

10.-30. (canceled)

31. The apparatus of claim 5, wherein the DNA origami linker further includes:
a plurality of scaffolding sections configured to organize into a predetermined structure; and
a plurality of staples configured to attach the plurality of scaffolding sections to one another, the plurality of staples including the plurality of binding sites.

32. The apparatus of claim 31, wherein the plurality of functional groups is a first plurality of functional groups, the DNA origami linker further including:
a second plurality of functional groups coupled to nucleotides of the DNA origami linker, the second plurality of functional groups configured to increase conductivity through the DNA origami linker.

33. The apparatus of claim 32, wherein the second plurality of functional groups are coupled to nucleotides within the plurality of binding sites.

34. The apparatus of claim 32, wherein the second plurality of functional groups include aromatic rings coupled to nucleotides of the DNA origami linker such that a pi orbital of aromatic rings are oriented to form an electron pathway between the electrode and the molecular wire.

35. The apparatus of claim 1, wherein the first electrode and the second electrode are disposed on a semiconductor chip including a plurality of electrodes, the semiconductor chip further including a plurality of molecular wires configured to electrically connect one or more sets of electrodes of the plurality of electrodes to form one or more circuits.

36. The apparatus of claim 35, wherein at least a portion of the plurality of molecular wires are configured to be replaced via toehold strand displacement such that the one or more circuits formed by the plurality of molecular wires can be regenerated or reprogrammed.

37. The apparatus of claim 35, wherein the one or more circuits includes a plurality of circuits configured to perform molecular sensing.

38. The apparatus of claim 35, wherein the one or more circuits includes a plurality of circuits configured to perform DNA sequencing.

39. The apparatus of claim 38, wherein the plurality of circuits are configured to operate in parallel.

40. The apparatus of claim 38, wherein the plurality of circuits are distributed across the semiconductor chip with a pitch of about 200 nm to about 500 nm.
